(19) United States
(12) Patent Application Publication
(10) Pub. No.: US 2024/0209459 A1
(43) Pub. Date: Jun. 27, 2024

(54) PERIANAL SWAB PCR PANEL, METHODS, DEVICE, AND KITS

(71) Applicant: IDEXX Laboratories, Inc., Westbrook, ME (US)

(72) Inventors: Melissa Jane Beall, Cape Elizabeth, ME (US); Phyllis Ione Tyrrell, Falmouth, ME (US)

(21) Appl. No.: 18/391,216

(22) Filed: Dec. 20, 2023

Related U.S. Application Data
(60) Provisional application No. 63/434,621, filed on Dec. 22, 2022.

(51) Int. Cl. C12Q 1/6893; C12Q 1/6844

(52) U.S. Cl. CPC C12Q 1/6893; C12Q 1/6844; C12Q 2600/16

(57) ABSTRACT

Methods, kit, and primer/probe sets are provided for the differential species-specific detection of one or more Dipylidium caninum, Toxocara canis, Ancylostoma canimum, Cystoisospora canis, Trichuris vulpis or Giardia duodenalis in an animal host based on amplification and detection of target parasite-specific repetitive DNA elements via polymerase chain reaction.

### CROSS-REFERENCE

The present application claims the benefit of priority from U.S. provisional application No. 63/434,621, filed Dec. 22, 2022, which is incorporated by reference in its entirety.

### FIELD OF THE DISCLOSURE

The present disclosure relates to a perianal swab PCR panel, compositions, devices, kits and methods for the detection of and distinguishing between intestinal parasite species in mammals. More particularly, the present disclosure relates to a PCR panel, devices, kits, and PCR-based methods for detecting the presence or absence of intestinal parasite species in perianal swab sample taken from a mammal and for distinguishing between worm nucleic acids, particularly at a veterinary or clinical setting.

### STATEMENT REGARDING SEQUENCE LISTING

A computer readable form of the Sequence Listing is filed with this application by electronic submission and is incorporated into this application by reference in its entirety. The sequence listing submitted herewith is contained in the text file created Dec. 20, 2023, entitled “22-2184-US.xml” and 19 kilobytes in size.

### BACKGROUND OF THE DISCLOSURE

Intestinal parasitic worm infections are common in animals and, if not diagnosed and treated, can cause serious disease or death. Current methods for diagnosis of parasitic worm infections primarily involve microscopic examination of fecal samples, either directly in fecal smears or following concentration of ova and parasites by flotation in density media or by sedimentation. Despite this procedure's high adoption, the method has significant shortcomings. These microscopic methods are time consuming and require specialized equipment. In addition, the accuracy of results of these methods is highly dependent upon the skill and expertise of the operator. For example, the presence of tapeworm is determined by looking for egg packets or proglottids, but these are excreted intermittently and in small numbers. Not surprisingly, tapeworm infection is often not diagnosed on routine fecal examination.

Stool handling is disagreeable and hazardous. Sanitary and inoffensive procedures for processing stool are awkward and often complex. Such procedures may include weighing, centrifuging and storing, and are difficult except in a clinical laboratory equipped with a suitable apparatus, protective equipment, and a skilled technician. Therefore, any reduction in the number of steps required to perform a fecal test at the point of care and any reduction in contact between test operator and the test material is desirable. Clinical laboratories have been using the immunoassay methods for the detection of various viruses, bacteria and non-helminth parasites and organisms in feces. However, such methods may not detect very low levels of parasites in miniscule samples such as a perianal swab or until a sufficient time as passed until the parasite reaches sexual maturity, resulting in a delay in detection and subsequent treatment.

Accordingly, there remains a need for a sensitive species-specific method for detecting parasitic infections at very low levels or pre-patent infections, e.g., infections prior to the time during which parasitic infections can be detected in a veterinary or clinical setting. Such a method would ideally detect parasitic worm infection in miniscule samples from infected mammal, particularly from the perianal area, at the point of care.

### SUMMARY OF THE DISCLOSURE

The present disclosure is generally directed to a perianal swab PCR panel, methods, kits, oligonucleotide primers and probes, primer/probe sets including the oligonucleotide primers and probes, for detecting and distinguishing between one or more different parasites in a sample containing biological material obtained from a mammal, e.g. a canine. In particular, the present disclosure relates to a highly sensitive species-specific PCR assay developed for detecting at least one or more genetic targets including the following: Dipylidium caninum, Toxocara canis. Ancylostoma canimum, Cystoisospora canis, Trichuris vulpis and Giardia duodenalis (also known as G. intestinalis or G. lamblia) at any life cycle stage and from samples including miniscule amounts (as low as 0.1 femtograms) of nucleic acid such as perianal swabs. The assay relies on target repetitive elements of nuclear or mitochondrial DNAs unique to each parasite as targets for detecting and distinguishing between one or more, two or more, three or more, or four or more parasites in a sample.

In accordance with one aspect of the disclosure, methods are provided for detecting and diagnosing a parasite infection in an animal host using PCR, using a repetitive sequence in DNA of the parasite in a sample obtained from the infected animal host. The sample can be biological material obtained from perianal area of the animal host. The term “perianal” refers to the area of the body surrounding the anus. The biological material can be obtained by any suitable means including a swab. The samples are generally obtained at the point of care, e.g., veterinary office or clinic.

In one embodiment, a method is provided for detecting at least one or more intestinal parasites selected from Dipylidium caninum, Toxocara canis, Ancylostoma caninum, Cystoisospora canis, Trichuris vulpis and Giardia duodenalis from a sample taken from a canine subject, the method comprising (a) providing a sample comprising biological material taken from the subject or nucleic acids extracted from the biological material taken from the subject, wherein the sample includes at least 0.1 femtograms of nucleic acids; (b) providing a composition suitable for detecting the one or more intestinal parasites selected from Dipylidium caninum, Toxocara canis, Ancylostoma canimum, Cystoisospora canis, Trichris vulpis and Giardia duodenalis, the composition comprising one or more of an oligonucleotide primer pair comprising forward and reverse primers directed to repetitive elements of Dipylidium canimum, Toxocara canis, Ancylostoma caninum, Cystoisospora canis, Trichuris vulpis or Giardia duodenalis, (c) contacting the sample with the composition of step (b) under conditions suitable for specific hybridization and extension of hybridized nucleic acids by polymerase chain reaction (PCR) to produce one or more PCR extension products, each PCR extension product associated with Dipylidium caninum, Toxocara canis, Ancylostoma canimum, Cystoisospora canis, Trichuris vulpis or Giardia duodenalis; (d) detecting for the one or more PCR extension products as an indication of the presence or absence of one or more intestinal parasites selected from Dipylidium caninum, Toxocara canis, Ancylostoma caninum, Cystoisospora canis, Trichuris vulpis and Giardia duodenalis, and (e) determining the presence of Dipylidium canimum in the sample if a PCR extension product associated with Dipylidium canimum is detected: the presence of Toxocara canis in the sample if a PCR extension product associated with Toxocara canis is detected: the presence of Ancylostoma caninum in the sample if a PCR extension product associated with Ancylostoma canimum is detected; the presence of Cystoisospora canis in the sample if a PCR extension product associated with Cystoisospora canis is detected, the presence of Trichuris vulpis in the sample if a PCR extension product associated with Trichuris vulpis is detected; and/or the presence of Giardia duodenalis in the sample if a PCR extension product associated with Giardia duodenalis is detected. The method beneficially allows for simultaneous detection of one or more intestinal parasites or nucleic acids thereof in a sample.

In some embodiments, the sample includes at least two or more, three or more or four or more intestinal parasites or nucleic acids thereof.

In some embodiments, the composition further comprises one or more detection probes, each probe specific for the PCR extension product and complementary to the repetitive DNA element uniquely possessed by the parasite. The detection probe can be a hybridization probe or a hydrolysis probe. In some embodiments, the one or more detection probes comprises one or more detectable labels selected from the group consisting of radioactive, calorimetric, fluorometric, luminescent and organic labels. In one embodiment, the detectable label comprises a fluorometric label.

In embodiments, the repetitive elements are present in low, medium or high copy number and are located in at least one of the nuclear genome and the mitochondrial genome of the parasite.

In some embodiments, the PCR is real-time PCR using a primer/probe set for each parasite. The parasite can be Dipylidium caninum, Toxocara canis, Ancylostoma caninum, Cystoisospora canis, Trichuris vulpis or Giardia duodenalis. In some embodiments, the parasite infection is patent. In other embodiments, the parasite infection is pre-patent. The sample may be obtained by a perianal swab from an infected mammalian host, e.g., canine, or nucleic acids extracted from the perianal swab.

In an embodiment, the method for detecting and diagnosing parasite Ancylostoma canimum involves amplification and detection of target the nuclear ITS1-5.8S ribosomal DNA (“rDNA”) of Ancylostoma canimum using an rDNA primer/probe set.

In an embodiment, the method for detecting and diagnosing Trichuris vulpis is based on amplification and detection of target 5.8S rDNA of Trichuris vulpis using a rDNA primer/probe set.

In an embodiment, the method for detecting and diagnosing Toxocara canis is based on amplification and detection of target mitochondrial NADH subunit 4 of Toxocara canis using a primer/probe set.

In an embodiment, the method for detecting and diagnosing Ancylostoma caninum is based on amplification of target mitochondrial NADH subunit 5 of Ancylostoma caninum utilizing an NADH primer/probe set.

In an embodiment, the method for detecting and diagnosing Dipylidium caninum is based on amplification and detection of target 28S rDNA of Dipylidium caninum utilizing a rDNA primer/probe set.

In an embodiment, method for detecting and diagnosing Cystoisospora canis is based on amplification and detection of target ITS1 rDNA of Cystoisospora canis utilizing a rDNA primer/probe set.

In accordance with another aspect of the disclosure, a method is provided for detecting and treating a mammalian subject infected with one or more intestinal parasites. In an embodiment, a method is provided for detecting one or more intestinal parasites selected from Dipylidium caninum, Toxocara canis, Ancylostoma canimum, Cystoisospora canis, Trichuris vulpis, and Giardia duodenalis from a sample taken from a canine subject and treating the canine subject is provided. The method comprising: (a) providing a sample comprising biological material taken from the subject or nucleic acids extracted from the biological material taken from the subject, wherein the sample includes at least 0.1 femtograms of nucleic acids; (b) providing a composition suitable for detecting the one or more intestinal parasites selected from Dipylidium caninum, Toxocara canis, Ancylostoma caninum, Cystoisospora canis, Trichuris vulpis and Giardia duodenalis, the composition comprising one or more of an oligonucleotide primer pair comprising forward and reverse primers directed to repetitive elements of Dipylidium canimum, Toxocara canis, Ancylostoma canimum, Cystoisospora canis, Trichuris vulpis or Giardia duodenalis; (c) contacting the sample with the composition of step (b) under conditions suitable for specific hybridization and extension of hybridized nucleic acids by polymerase chain reaction (PCR) to produce one or more PCR extension products, each PCR extension product associated with Dipylidium caninum, Toxocara canis, Ancylostoma canimum, Cystoisospora canis, Trichuris vulpis or Giardia duodenalis; (d) detecting for the one or more PCR extension products as an indication of the presence or absence of one or more intestinal parasites selected from Dipylidium canimum, Toxocara canis, Ancylostoma canimum, Cystoisospora canis, Trichuris vulpis and Giardia duodenalis; (e) determining the presence of Dipylidium caninum in the sample if a PCR extension product associated with Dipylidium caninum is detected; the presence of Toxocara canis in the sample if a PCR extension product associated with Toxocara canis is detected; the presence of Ancylostoma canimum in the sample if a PCR extension product associated with Ancylostoma canimum is detected, the presence of Cystoisospora canis in the sample if a PCR extension product associated with Cystoisospora canis is detected; the presence of Trichuris vulpis in the sample if a PCR extension product associated with Trichris vulpis is detected; and/or the presence of Giardia duodenalis in the sample if a PCR extension product associated with Giardia duodenalis is detected; and (f) if Dipylidium caninum, Toxocara canis, Ancylostoma canimum, Cystoisospora canis, Trichuris vulpis and/or Giardia duodenalis is detected, administering to the canine subject a treatment effective at reducing or eliminating the parasite infection. The method can beneficially allow for simultaneous detection of one or more, two or more, three or more intestinal parasites or nucleic acids thereof in a sample.

In another aspect of the disclosure, a kit is provided for detecting one or more intestinal parasites selected from Dipylidium caninum, Toxocara canis, Ancylostoma canimum, Cystoisospora canis, Trichuris vulpis and Giardia duodenalis from a sample taken from a canine subject. The kit comprising (i) one or more oligonucleotide primer pairs comprising of forward and reverse primers directed to repetitive elements of Dipylidium canimum, Toxocara canis, Ancylostoma caninum, Cystoisospora canis, Trichuris vulpis or Giardia duodenalis; (ii) detection probes for detecting PCR extension products associated with the one or more intestinal parasites selected from Dipylidium caninum, Toxocara canis, Ancylostoma canimum, Cystoisospora canis, Trichuris vulpis and Giardia duodenalis; and (iii) optional buffers and a set of instructions.

In some embodiments, the kit includes one or more of the primer/probe sets as described above.

### BRIEF DESCRIPTION OF THE DRAWINGS

The foregoing features of embodiments will be more readily understood by reference to the following detailed description, taken with reference to the accompanying drawings.

FIG. 1 shows a table of the results of multiplex PCR assay carried out to evaluate the performance of individual low/moderately repetitive copy target ribosomal genes as well as high copy target mitochondrial genes. The table includes the mean crossing points (CP) for each assay at each plasmid amount. Each assay was able to detect its Target Plasmid at a plasmid amount as low as 0.1 femtogram (fg). The negative control is indicated by “nc” (i.e., no Target Plasmid).

FIG. 2 shows a table of the results of multiplex PCR assays carried out to verify and evaluate the performance of samples including dilutions of A. canimum (low copy target), T. canis (high copy target), and T. vulpis (low copy target) using hybridization detection probes. The table includes the mean crossing points (CP) for each assay at each plasmid amount. All samples contained canine swab DNA. The negative control indicated by “swab extract” did not contain any Target Plasmid. The negative control indicated by “nc” contained neither Target Plasmid nor canine swab DNA. Each assay was able to detect its Target Plasmid at a plasmid amount as low as 0.1 fg in the presence of canine swab DNA.

FIG. 3 shows a side-by side comparison of the CP values shown in FIG. 1 and FIG. 2. In each case, the CP values obtained by amplification in the presence of canine swab DNA closely track the CP values obtained by amplification in the absence of canine swab DNA. Therefore, assay sensitivity was not affected by the presence of multiple targets and background sample DNA.

FIG. 4 shows the results of multiplex real time PCR assays using hydrolysis detection probes and performance of individual low/moderately repetitive copy target ribosomal genes as well as high copy target mitochondrial genes. Sensitivity of individual targets is not affected by multiplex format. The table in FIG. 4 shows the mean crossing points (CP) for each assay at each plasmid amount. Each assay was able to detect its Target Plasmid at a plasmid amount as low as 0.1 fg. The negative control is indicated by “nc” (i.e., no Target Plasmid). Therefore, the limit of detection was at least as low as 0.1 fg of each plasmid. The CP values did not indicate that combining the three primer/probe sets affected the limit of detection.

FIG. 5 includes a table of the results of multiplex PCR assays carried out to verify and evaluate the performance of samples including dilutions of A. caninum (high copy target), D. caninum (low copy target), and C. canis (low copy target) using hydrolysis detection probes. The table includes the mean crossing points (CP) for each assay at each plasmid amount. All samples contained canine swab DNA. The negative control indicated by “nc” did not contain any Target Plasmid or canine swab DNA. Each assay was able to detect its Target Plasmid at a plasmid amount as low as 0.1 fg in the presence of canine swab DNA. Therefore, the limit of detection was at least as low as 0.1 fg of each plasmid in the presence of canine swab DNA.

FIG. 6 shows a side-by side comparison of the CP values shown in FIG. 4 and FIG. 5. In each case, the CP values obtained by amplification in the presence of canine swab DNA closely track the CP values obtained by amplification in the absence of canine swab DNA. Therefore, assay sensitivity was not affected by the presence of multiple targets and background sample DNA.

### SUMMARY OF THE DISCLOSURE

In one aspect of the disclosure, a method for detecting at least one or more intestinal parasites selected from Dipylidium caninum, Toxocara canis, Ancylostoma canimum, Cystoisospora canis, Trichuris vulpis and Giardia duodenalis from a sample taken from a canine subject is provided. The method includes: (a) providing a sample comprising biological material taken from the subject or nucleic acids extracted from the biological material taken from the subject, wherein the sample includes at least 0.1 femtograms of nucleic acids, (b) providing a composition suitable for detecting the one or more intestinal parasites selected from Dipylidium caninum, Toxocara canis, Ancylostoma caninum, Cystoisospora canis, Trichuris vulpis and Giardia duodenalis, the composition comprising one or more of an oligonucleotide primer pair comprising forward and reverse primers directed to repetitive elements of Dipylidium canimum, Toxocara canis, Ancylostoma canimum, Cystoisospora canis, Trichuris vulpis or Giardia duodenalis; (c) contacting the sample with the composition of step (b) under conditions suitable for specific hybridization and extension of hybridized nucleic acids by polymerase chain reaction (PCR) to produce one or more PCR extension products, each PCR extension product associated with Dipylidium canimum, Toxocara canis, Ancylostoma caninum, Cystoisospora canis, Trichris vulpis or Giardia duodenalis; (d) detecting the one or more PCR extension products as an indication of the presence or absence of one or more intestinal parasites selected from Dipylidium caninum, Toxocara canis, Ancylostoma canimum, Cystoisospora canis, Trichris vulpis and Giardia duodenalis; and (e) determining the presence of Dipylidium canimum in the sample if a PCR extension product associated with Dipylidium canimum is detected; the presence of Toxocara canis in the sample if a PCR extension product associated with Toxocara canis is detected; the presence of Ancylostoma canimum in the sample if a PCR extension product associated with Ancylostoma canimum is detected; the presence of Cystoisospora canis in the sample if a PCR extension product associated with Cystoisospora canis is detected; the presence of Trichuris vulpis in the sample if a PCR extension product associated with Trichuris vulpis is detected; and/or the presence of Giardia duodenalis in the sample if a PCR extension product associated with Giardia duodenalis is detected.

In another aspect, a method is provided for detecting at least one or more intestinal parasites selected from Dipylidium canimum, Toxocara canis, Ancylostoma canimum, Cystoisospora canis, Trichuris vulpis and Giardia duodenalis from a sample taken from a canine subject and treating the canine subject, the method including: (a) providing a sample comprising biological material taken from the subject or nucleic acids extracted from the biological material taken from the subject, wherein the sample includes at least 0.1 femtograms of nucleic acids; (b) providing a composition suitable for detecting the one or more intestinal parasites selected from Dipylidium canimum, Toxocara canis, Ancylostoma caninum, Cystoisospora canis, Trichuris vulpis and Giardia duodenalis, the composition comprising one or more of an oligonucleotide primer pair comprising forward and reverse primers directed to repetitive elements of Dipylidium canimum, Toxocara canis, Ancylostoma caninum, Cystoisospora canis, Trichuris vulpis or Giardia duodenalis; (c) contacting the sample with the composition of step (b) under conditions suitable for specific hybridization and extension of hybridized nucleic acids by polymerase chain reaction (PCR) to produce one or more PCR extension products, each PCR extension product associated with Dipylidium canimum, Toxocara canis, Ancylostoma canimum, Cystoisospora canis, Trichuris vulpis or Giardia duodenalis; (d) detecting for the one or more PCR extension products as an indication of the presence or absence of one or more intestinal parasites selected from Dipylidium canimum, Toxocara canis, Ancylostoma canimum, Cystoisospora canis, Trichuris vulpis and Giardia duodenalis; and (e) determining the presence of Dipylidium canimum in the sample if a PCR extension product associated with Dipylidium caninum is detected; the presence of Toxocara canis in the sample if a PCR extension product associated with Toxocara canis is detected; the presence of Ancylostoma canimum in the sample if a PCR extension product associated with Ancylostoma caninum is detected, the presence of Cystoisospora canis in the sample if a PCR extension product associated with Cystoisospora canis is detected; the presence of Trichuris vulpis in the sample if a PCR extension product associated with Trichuris vulpis is detected; and/or the presence of Giardia duodenalis in the sample if a PCR extension product associated with Giardia duodenalis is detected; and (f) if the presence of one or more Dipylidium canimum, Toxocara canis, Ancylostoma canimum, Cystoisospora canis, Trichuris vulpis and/or Giardia duodenalis is detected, administering to the canine subject a treatment effective at reducing or eliminating parasite infection.

In some embodiments of the above methods, the sample includes two or more, three or more or four or more intestinal parasites or nucleic acids thereof.

In some embodiments of the above methods, the composition further comprises one or more detection probes, each probe specific for the PCR extension product.

In some embodiments of the above methods, the detection probe is a hybridization probe or a hydrolysis probe.

In some embodiments of the above methods, the one or more detection probes comprises a detectable label selected from the group consisting of radioactive, calorimetric, fluorometric, luminescent and organic labels.

In some embodiments of the above methods, the detectable label comprises a fluorometric label.

In some embodiments of the above methods, the biological material is collected from the perianal area of the subject.

In some embodiments of the above methods, the biological material is collected with a swab.

In some embodiments of the above methods, the sample is collected at the point of care.

In some embodiments of the above methods, step (c) is carried out at the point of care.

In some embodiments of the above methods, the repetitive elements are present in low, medium or high copy number.

In some embodiments of the above methods, the repetitive elements are located in at least one of the nuclear genome and the mitochondrial genome.

In some embodiments of the above methods, steps (c) and (d) are carried out using one or more oligonucleotide primer/probe sets selected from the group consisting of:

(a)
a 17-mer oligonucleotide forward primer
(SEQ ID NO: 1)
5′ GGCACCTGTCTGTCAGG 3′
and
19-mer reverse primer
(SEQ ID NO: 2)
5′ TCTAAGCGTCTGCAATTCG 3′,
a labeled 27-mer donor probe
(SEQ ID NO: 3)
5′ ACGTTTAATGTTTGCAGAATCGTGACT-FL 3′
and
a labeled 24-mer acceptor probe
(SEQ ID NO: 4)
5′ LC640-ACCTAGCTTCAGCGATGGATCGGT-PH 3′;
(b)
a 17-mer oligonucleotide forward primer
(SEQ ID NO: 5)
5′ TTCCGAACGGCGGATCA 5′
and
16-mer reverse primer
(SEQ ID NO: 6)
5′ CTCAGACAGGCGTAGC 3′,
a labeled 30-mer donor probe
(SEQ ID NO: 7)
5′ TGATGTGAATTGCAGACACACTGAACTTGA-FL 3′
and
a labeled 26-mer acceptor probe
(SEQ ID NO: 8)
5′ LC610-TACTTTGAACGCACATTGCAGCGTCG-PH 3′;
(c)
a 21-mer oligonucleotide forward primer
(SEQ ID NO: 9)
5′ TCTTGCATTTGTGGTTGCCTA 3′
and
22-mer reverse primer
(SEQ ID NO: 10)
5′ GCATCACTCTGAAATACACAAC 3′,
labeled 16-mer donor probe
(SEQ ID NO: 11)
5′ GTTGAGGCCCCCACGA-FL 3′
and
labeled 24-mer acceptor probe
(SEQ ID NO: 12)
5′ LC670-CCAGTATGTTGTTGGCTGGTCTTT-PH 3′;
(d)
a 26-mer oligonucleotide forward primer
(SEQ ID NO: 13)
5′ CTTTTAGAAGATGATTACCTAAGGCT 3′
and
21-mer reverse primer
(SEQ ID NO: 14)
5′ ACGTTCTACTATGAACCAACG 3′,
and
a 20-mer probe
(SEQ ID NO: 15)
5′ LC640-AGTGCGCCGACGCCTGTTAG-BBQ 3′ ;
(e)
a 17-mer oligonucleotide forward primer
(SEQ ID NO: 16)
5′ GATCCCGTTGTTAGGCA 3′
and
17-mer reverse primer
(SEQ ID NO: 17)
5′ TCGATGACCACACCATG 3′,
and
a labeled 14-mer probe
(SEQ ID NO: 18)
5′ 6-FAM-GTGTGTGCACAGTC-MGB-NFQ 3′;
and
(f)
a 21-mer oligonucleotide forward primer
(SEQ ID NO: 19)
5′ ATTCTCTGCTATTTGGTGACG 3′
and
17-mer reverse primer
(SEQ ID NO: 20)
5′ ACCTTCAGCAACAAGGC 3′,
and
a labeled 26-mer probe
(SEQ ID NO: 21)
5′ LC670-ATGCCCATAGCCAAAGAGAGTCCACG-BBQ 3′.

In another aspect of the invention, a kit is provided for detecting one or more intestinal parasites selected from Dipylidium canimum, Toxocara canis, Ancylostoma caninum, Cystoisospora canis, Trichuris vulpis and Giardia duodenalis from a sample taken from a canine subject. The kit comprises: (i) one or more oligonucleotide primer pairs comprising of forward and reverse primers directed to repetitive elements of Dipylidium caninum, Toxocara canis, Ancylostoma caninum, Cystoisospora canis, Trichuris vulpis or Giardia duodenalis: (ii) detection probes for detecting PCR extension products associated with the one or more intestinal parasites selected from Dipylidium caninum, Toxocara canis, Ancylostoma caninum, Cystoisospora canis, Trichuris vulpis and Giardia duodenalis; and (iii) optional buffers and a set of instructions.

In some embodiments, parts (i) and (ii) are part of an oligonucleotide primer/probe set, the oligonucleotide primer/probe set selected from the group consisting of:

(a)
a 17-mer oligonucleotide forward primer
(SEQ ID NO: 1)
5′ GGCACCTGTCTGTCAGG 3′
and
19-mer reverse primer
(SEQ ID NO: 2)
5′ TCTAAGCGTCTGCAATTCG 3′,
a labeled 27-mer donor probe
(SEQ ID NO: 3)
5′ ACGTTTAATGTTTGCAGAATCGTGACT-FL 3′
and
a labeled 24-mer acceptor probe
(SEQ ID NO: 4)
5′ LC640-ACCTAGCTTCAGCGATGGATCGGT-PH 3′;
(b)
a 17-mer oligonucleotide forward primer
(SEQ ID NO: 5)
5′ TTCCGAACGGCGGATCA 5′
and
16-mer reverse primer
(SEQ ID NO: 6)
5′ CTCAGACAGGCGTAGC 3′,
a labeled 30-mer donor probe
(SEQ ID NO: 7)
5′ TGATGTGAATTGCAGACACACTGAACTTGA-FL 3′
and
a labeled 26-mer acceptor probe
(SEQ ID NO: 8)
5′ LC610-TACTTTGAACGCACATTGCAGCGTCG-PH 3′;
(c)
a 21-mer oligonucleotide forward primer
(SEQ ID NO: 9)
5′ TCTTGCATTTGTGGTTGCCTA 3′
and
22-mer reverse primer
(SEQ ID NO: 10)
5′ GCATCACTCTGAAATACACAAC 3′,
labeled 16-mer donor probe
(SEQ ID NO: 11)
5′ GTTGAGGCCCCCACGA-FL 3′
and
labeled 24-mer acceptor probe
(SEQ ID NO: 12)
5′ LC670-CCAGTATGTTGTTGGCTGGTCTTT-PH 3′;
(d)
a 26-mer oligonucleotide forward primer
(SEQ ID NO: 13)
5′ CTTTTAGAAGATGATTACCTAAGGCT 3′
and
21-mer reverse primer
(SEQ ID NO: 14)
5′ ACGTTCTACTATGAACCAACG 3′,
and
a 20-mer probe
(SEQ ID NO: 15)
5′ LC640-AGTGCGCCGACGCCTGTTAG-BBQ 3′;
(e)
a 17-mer oligonucleotide forward primer
(SEQ ID NO: 16)
5′ GATCCCGTTGTTAGGCA 3′
and
17-mer reverse primer
(SEQ ID NO: 17)
5′ TCGATGACCACACCATG 3′,
and
a labeled 14-mer probe
(SEQ ID NO: 18)
5′ 6-FAM-GTGTGTGCACAGTC-MGB-NFQ 3′;
and
(f)
a 21-mer oligonucleotide forward primer
(SEQ ID NO: 19)
5′ ATTCTCTGCTATTTGGTGACG 3′
and
17-mer reverse primer
(SEQ ID NO: 20)
5′ ACCTTCAGCAACAAGGC 3′,
and
a labeled 26-mer probe
(SEQ ID NO: 21)
5′ LC670-ATGCCCATAGCCAAAGAGAGTCCACG-BBQ 3′.

In another aspect, an oligonucleotide primer/probe set selected from the group consisting of:

(a)
a 17-mer oligonucleotide forward primer
(SEQ ID NO: 1)
5′ GGCACCTGTCTGTCAGG 3′
and
19-mer reverse primer
(SEQ ID NO: 2)
5′ TCTAAGCGTCTGCAATTCG 3′,
a labeled 27-mer donor probe
(SEQ ID NO: 3)
5′ ACGTTTAATGTTTGCAGAATCGTGACT-FL 3′
and
a labeled 24-mer acceptor probe
(SEQ ID NO: 4)
5′ LC640-ACCTAGCTTCAGCGATGGATCGGT-PH 3′;
(b)
a 17-mer oligonucleotide forward primer
(SEQ ID NO: 5)
5′ TTCCGAACGGCGGATCA 5′
and
16-mer reverse primer
(SEQ ID NO: 6)
5′ CTCAGACAGGCGTAGC 3′,
a labeled 30-mer donor probe
(SEQ ID NO: 7)
5′ TGATGTGAATTGCAGACACACTGAACTTGA-FL 3′
and
a labeled 26-mer acceptor probe
(SEQ ID NO: 8)
5′ LC610-TACTTTGAACGCACATTGCAGCGTCG-PH 3′;
(c)
a 21-mer oligonucleotide forward primer
(SEQ ID NO: 9)
5′ TCTTGCATTTGTGGTTGCCTA 3′
and
22-mer reverse primer
(SEQ ID NO: 10)
5′ GCATCACTCTGAAATACACAAC 3′,
labeled 16-mer donor probe
(SEQ ID NO: 11)
5′ GTTGAGGCCCCCACGA-FL 3′
and
labeled 24-mer acceptor probe
(SEQ ID NO: 12)
5′ LC670-CCAGTATGTTGTTGGCTGGTCTTT-PH 3′;
(d)
a 26-mer oligonucleotide forward primer
(SEQ ID NO: 13)
5′ CTTTTAGAAGATGATTACCTAAGGCT 3′
and
21-mer reverse primer
(SEQ ID NO: 14)
5′ ACGTTCTACTATGAACCAACG 3′,
and
a 20-mer probe
(SEQ ID NO: 15)
5′ LC640-AGTGCGCCGACGCCTGTTAG-BBQ 3′;
(e)
a 17-mer oligonucleotide forward primer
(SEQ ID NO: 16)
5′ GATCCCGTTGTTAGGCA 3′
and
17-mer reverse primer
(SEQ ID NO: 17)
5′ TCGATGACCACACCATG 3′,
and
a labeled 14-mer probe
(SEQ ID NO: 18)
5′ 6-FAM-GTGTGTGCACAGTC-MGB-NFQ 3′;
and
(f)
a 21-mer oligonucleotide forward primer
(SEQ ID NO: 19)
5′ ATTCTCTGCTATTTGGTGACG 3′
and
17-mer reverse primer
(SEQ ID NO: 20)
5′ ACCTTCAGCAACAAGGC 3′,
and
a labeled 26-mer probe
(SEQ ID NO: 21)
5′ LC670-ATGCCCATAGCCAAAGAGAGTCCACG-BBQ 3′.

These and other embodiments of the disclosure will be apparent in light of the disclosure below.

### DETAILED DESCRIPTION OF DISCLOSURE

### Definitions

As used in this description and the accompanying claims, the following terms shall have the meanings indicated, unless the context otherwise requires:

A “set” includes at least one member.

As used herein, “real-time polymerase chain reaction,” “real-time PCR,” “quantitative polymerase chain reaction,” and “qPCR” are synonymous.

As used herein, “infected,” e.g., an infected animal host, or an infected mammalian host, shall mean that an organism harbors parasite.

“Pre-patent infections,” and the like, shall mean one or more Dipylidium canimum, Toxocara canis, Ancylostoma canimum, Cystoisospora canis, and/or Trichuris vulpis infections prior to the time during which such infections produce eggs or cysts or oocysts or proglottids or egg packets that are detectable in fecal material of the host.

“Patent infections,” and the like, shall mean Dipylidium canimum, Toxocara canis, Ancylostoma canimum, Cystoisospora canis, and/or Trichuris vulpis infections occurring after the time during which one or more Dipylidium canimum, Toxocara canis, Ancylostoma canimum, Cystoisospora canis, and/or Trichuris vulpis infections produce eggs or cysts or oocysts or proglottids or egg packets that are detectable in fecal material of the host.

“Mammalian host” shall mean a mammal that is infected with a parasite, including canines.

“Complementary” shall mean that at least 80% of the bases of an oligonucleotide, primer, or probe are capable of base pairing with a target sequence.

“Treatment” means the administration of a therapeutic agent to a patient by any suitable administration route to reduce or eliminate one or more Dipylidium canimum, Toxocara canis, Ancylostoma caninum, Cystoisospora canis, Trichuris vulpis and/or Giardia duodenalis infections from the body either by controlling, stunning or killing them and/or that reduces or eliminates infestation by intermediate hosts such as insects, e.g. fleas, which can transmit infection with parasitic worms or other internal parasites, without causing significant damage to the patient.

“Nucleic acid” is synonymous with, and therefore is used interchangeably with, “gene”, “DNA”, “cDNA”, “EST”, “polynucleotide”, “oligonucleotide”, “polynucleic acid”, “RNA” and “mRNA”. A nucleic acid may be in double-stranded form or it may be in single-stranded form. Further, a nucleic acid is either naturally isolated, such as from a whole parasite or a portion thereof, for example, or it is artificially synthesized, either in a recombinant host organism or by any other artificial means known to the skilled artisan, such as by employing a PCR-based technique, by creating a transgenic organism that synthesizes the nucleic acid, by using a DNA synthesizing machine, or by any another molecular-based technique, for example.

The present disclosure is generally directed to methods, kits, oligonucleotide primers and probes, primer/probe sets including the oligonucleotide primers and probes, for detecting and distinguishing between one or more different parasites in a sample containing biological material obtained from a mammal, e.g. canine. In particular, the present disclosure relates to a highly sensitive species-specific PCR assay developed for detecting one or more genetic targets including at least one or more of the following: Dipylidium caninum, Toxocara canis, Ancylostoma canimum, Cystoisospora canis, Trichuris vulpis and/or Giardia duodenalis at any life cycle stage and from samples including miniscule amounts of biological material as perianal swabs. The assay relies on target repetitive elements of nuclear (ribosomal) or mitochondrial DNAs unique to each parasite as targets for detecting and distinguishing between two or more parasites in a sample.

The collection of biological material by perianal swab are widely known to those skilled in the art. Procedures and methods for the collection and handling of biological material by perianal swab are described in Little et al., Diagnosis of canine intestinal parasites: Improved detection of Dipylidium canimum infection through coproantigen testing. Vet Parasitol. 2023 December; 324:110073. doi: 10.1016/j.vetpar.2023.110073, and in Labuschagne et al., Analysis of Dipylidium canimum tapeworms from dogs and cats, or their respective fleas—Part 1. Molecular characterization of Dipylidium canimum: genetic analysis supporting two distinct species adapted to dogs and cats. Parasite. 2018; 25:30. doi: 10.1051/parasite/2018028 (both of which which are incorporated herein by reference by reference in their entirety). The perianal swab collection can be performed by contacting the anal region (which can include the surrounding hair) with a swab. In some embodiments, the swab can be a cotton swab. The swab can be sterile.

Described herein are platforms, including methods, kits, and primer/probe sets for the differential detection of one or more Dipylidium caninum, Toxocara canis, Ancylostoma common, Cystoisospora canis, Trichuris vulpis and/or Giardia duodenalis in a sample through the use of parasite-specific DNA target capture techniques and polymerase chain reaction (“PCR”) detection of those targets in accordance with embodiments of the present disclosure. In some embodiments, PCR is real-time PCR. Because of their high sensitivity and species-specificity, these platforms can be used to screen samples to detect infections of one or more of Dipylidium caninum, Toxocara canis, Ancylostoma canimum, Cystoisospora canis, Trichuris vulpis and/or Giardia duodenalis as early as 3 weeks post-infection and at detection limits of 0.1 femtograms (fg) of nucleic acid in the sample taken from the mammalian subject. In accordance with embodiments of the present disclosure, patent infections and pre-patent infections may be detected by allowing for the selective capture of genomic target DNA or cell-free target DNA.

In some embodiments, the platforms described herein are capable of detecting pre-patent infections of one or more of Dipylidium canimum, Toxocara canis, Ancylostoma canimum, Cystoisospora canis, Trichuris vulpis and/or Giardia duodenalis. The ability to detect pre-patent infections would be clinically useful, opening the door for new treatment strategies, because the infected animal may be treated early with a lower dose of medication and with less treatment side effects and less physiological damage due to the infection. Treating during the pre-patent period prevents environmental contamination and reduces transmission.

In some embodiments, the platforms described herein are intended for use by veterinarians and clinical laboratories to enable early detection of parasite infection. The platform targets genomic repetitive DNA elements that are both unique to the organism and present at greatest copy number within the parasite's genome. These DNA elements represent the optimal targets for the detection of their respective parasites because they are both unique to their species of origin and highly repetitive within their genome of origin, making them species-specific and optimally sensitive detection assay targets. These repetitive DNA elements are sensitive targets for DNA detection assays because the greater the number of targets, the more sensitive the assay By designing oligonucleotide constructs with sequence complementarity to these assay targets, oligonucleotides that are capable of binding their specific genomic target sequences may be used to capture DNA molecules comprising the identified target sequences.

The repetitive species-specific DNA sequences of each parasite used to design oligonucleotides primers and probes are described herein. Representative examples of oligonucleotides primers and probes are described in the Examples below.

In some embodiments, highly sensitive species-specific detection of parasites may be achieved by using PCR-based assays on the isolated DNA sample. PCR-based assays were developed by exploiting the same sensitive and species-specific repetitive DNA elements targeted for capture. In some embodiments, for each parasite, PCR primers were designed to amplify these target DNA sequences, and a modified real-time PCR probe construct was designed to enable detection (e.g., fluorescence or luminescence) following amplification.

In its most typical form, DNA exists as a double-stranded molecule consisting of a first strand and a second strand. The sequence of each of these strands is the reverse complement of the other and each strand is base-paired with the other strand. In embodiments, both strands of the repetitive species-specific target DNA are hybridized to oligonucleotide primers complementary to a sequence or sequences present on each strand, and are amplified and detected using PCR.

Generally, each PCR primer and probe ranges from 12 base pairs (bp) to 40 bp in length. Although primers and probes need not be perfectly complementary to their target sequences, at least 80% of the bases of an oligonucleotide, primer, or probe are capable of base pairing with a target sequence.

Various real-time PCR probes may be utilized, which comprise a fluorophore, i.e., a fluorescent dye, and one or two quenchers. In some embodiments, the probe design includes a pair of a donor oligonucleotide probe and an acceptor oligonucleotide probe, each probe labeled with member of a FRET pair. This probe pair is referred to as the hybridization probe in the Examples below. For instance, the donor probe may be labeled with FITC fluorophore, i.e., a fluorescent dye, linked to the 3′ end of the construct. The acceptor probe may be labeled at the 5′ end with LC® Red 640, LC® Red 610, or LC® Red 670 The acceptor probes may have a 3′ cap (noted as “PH” in the sequences). In other embodiments, the hydrolysis probe design includes an oligonucleotide probe conjugated to fluorescent labels and quenchers. For instance, the probe can be labeled at the 5′ end with LC@ Red 640 and LC® Red 670 or 6-FAM (6-carboxyflorescein) and at the 3′ end with BlackBerry quencher (BBQ) or non-fluorescent Quencher-Minor Groove Binder (MGB-FNQ).

Probes that utilize modified nucleotides may also be used. For example, Affinity Plus qPCR Probes (Integrated DNA. Technologies (Coralville, Iowa, USA)) incorporate several locked nucleic acid (LNA) nucleotides into the probe in order to give the probe higher structural stability and increased Tₘ, which may lead to greater specificity. In addition, PrimeTime LNA qPCR Probes (Integrated DNA Technologies (Coralville, Iowa, USA), which utilize one or more LNAs, may be used in order to give the probe higher structural stability for short probes and increased Tₘ, which may lead to greater specificity.

Molecular beacon real-time PCR probes may also be used. Molecular beacon probes have self-complementary ends that form a quenched, hairpin structure when not bound to their target sequence. Molecular beacon probes comprise a fluorophore at one end and a quencher at the other end. Fluorescent signal is generated by the linearization of the probe upon hybridization to its target sequence during real-time PCR cycling.

Exemplary sets of primer and probe constructs are described in the Examples. In an embodiment, the method for detecting and diagnosing parasite Ancylostoma canimum involves amplification and detection of target ITS1-5.8S ribosomal DNA (“rDNA”) of Ancylostoma canimum using an rDNA primer/probe set comprising a 17-mer oligonucleotide forward primer 5′ GGCACCTGTCTGTCAGG 3′ (SEQ ID NO: 1) and 19-mer reverse primer 5′ TCTAAGCGTCTGCAATTCG 3′ (SEQ ID NO: 2), a labeled 27-mer donor probe 5′ ACGTTTAATGTTTGCAGAATCGTGACT-FL 3′ (SEQ ID NO: 3) and a labeled 24-mer acceptor probe 5′ LC640-ACCTAGCTTCAGCGATGGATCGGT-PH 3′(SEQ ID NO: 4).

In an embodiment, the method for detecting and diagnosing Trichuris vulpis is based on amplification and detection of target 5.8S rDNA of Trichuris vulpis using a rDNA primer/probe set comprising a 17-mer oligonucleotide forward primer 5′ TTCCGAACGGCGGATCA 5′ (SEQ ID NO: 5) and 16-mer reverse primer 5′ CTCAGACAGGCGTAGC 3′ (SEQ ID NO: 6), a labeled 30-mer donor probe 5′ TGATGTGAATTGCAGACACACTGAACTTGA-FL 3′ (SEQ ID NO: 7) and a labeled 26-mer acceptor probe 5′ LC610-TACTTTGAACGCACATTGCAGCGTCG-PH 3′ (SEQ ID NO: 8).

In an embodiment, the method for detecting and diagnosing Toxocara canis is based on amplification and detection of target mitochondrial NADH subunit 4 of Toxocara canis using a primer/probe set comprising a 21-mer oligonucleotide forward primer 5′ TCTTGCATTTGTGGTTGCCTA 3′(SEQ ID NO: 9) and 22-mer reverse primer 5′ GCATCACTCTGAAATACACAAC 3′ (SEQ ID NO: 10), labeled 16-mer donor probe 5′ GTTGAGGCCCCCACGA-FL 3′ (SEQ ID NO: 11) and labeled 24-mer acceptor probe 5′ LC670-CCAGTATGTTGTTGGCTGGTCTTT-PH 3′ (SEQ ID NO: 12).

In an embodiment, the method for detecting and diagnosing Ancylostoma canimum is based on amplification of target mitochondrial NADH subunit 5 of Ancylostoma canimum utilizing an NADH primer/probe set comprising a 26-mer oligonucleotide forward primer 5′ CTTTTAGAAGATGATTACCTAAGGCT 3′ (SEQ ID NO: 13) and 21-mer reverse primer 5′ ACGTTCTACTATGAACCAACG 3′ (SEQ ID NO: 14), and a 20-mer probe 5′ LC640-AGTGCGCCGACGCCTGTTAG-BBQ 3′(SEQ ID NO: 15).

In an embodiment, the method for detecting and diagnosing Dipylidium canimum is based on amplification and detection of target 28S rDNA of Dipylidium canimum utilizing a rDNA primer/probe set comprising a 17-mer oligonucleotide forward primer 5′ GATCCCGTTGTTAGGCA 3′ (SEQ ID NO: 16) and 17-mer reverse primer 5′ TCGATGACCACACCATG 3′ (SEQ ID NO: 17), and a labeled 14-mer probe 5′ 6-FAM-GTGTGTGCACAGTC-MGB-NFQ 3′ (SEQ ID NO: 18).

In an embodiment, the method for detecting and diagnosing Cystoisospora canis is based on amplification and detection of target ITS1 rDNA of Cystoisospora canis utilizing a rDNA primer/probe set comprising a 21-mer oligonucleotide forward primer 5′ ATTCTCTGCTATTTGGTGACG 3′ (SEQ ID NO: 19) and 17-mer reverse primer 5′ ACCTTCAGCAACAAGGC 3′ (SEQ ID NO: 20), and a labeled 26-mer probe 5′ LC670-ATGCCCATAGCCAAAGAGAGTCCACG-BBQ 3′(SEQ ID NO: 21).

In some embodiments, the samples obtained from mammalian subjects, including potentially infected samples from dogs potentially harboring parasites, are processed to facilitate the release of pathogen-derived target DNA if present. Target DNA is then detected using PCR. The genomic target DNA that is present at all life stages, allowing for the detection of both pre-patent and patent infections.

In some embodiments, an optimal primer and probe set is provided that is effective in a real-time PCR assay for detecting the presence of target repetitive sequences from DNA of one or more Dipylidium canimum, Toxocara canis, Ancylostoma caninum, Cystoisospora canis, Trichuris vulpis and/or Giardia duodenalis in a sample obtained from a mammalian host. Detection of the target DNA for a specific parasite indicates that that the mammalian host is infected with that parasite. For example, in some embodiments, exemplary primer and probe set A c rDNA primer/probe set was used for amplification and detection of ITS1-5.8s rDNA of Ancylostoma canimum; Trich. rDNA primer/probe set was used for amplification and detection of 5.8S rDNA of Trichris vulpis; T.c NADH primer/probe set was used for amplification and detection of mitochondrial NADH submit 4 of Toxocara canis; D.c rDNA primer/probe set was utilized for amplification and detection of 28S rDNA of Dipylidium canimum; and C.c rDNA primer/probe set was used for amplification and detection of ITS1 rDNA of Cystoisospora canis. One or more of these primer/probe sets can be used for single or multiplex PCR assay for detecting at least one or more of Dipylidium caninum, Toxocara canis, Ancylostoma caninum, Cystoisospora canis, Trichuris vulpis and/or Giardia duodenalis.

In some embodiments, a method of treating a mammalian host infected with a parasite is provided. Patients suffering from infections with intestinal parasitic worms such as tapeworms (including Dipylidium canimum, Taema pisiformis and Taenia taeniaformus), hookworms (including Ancylostoma caninum), whipworms (including Trichuris vulpis) and/or roundworms (including Toxocara canis) can be treated with de-worming drugs, also known as anthelminties (or anthelminthics). Such anthelminties are widely known to those skilled in the art. Anthelmintics for the treatment of tapeworms include, without limitation praziquantel, nitazoxanide, albendazole, epsiprantel, fenbendazole, or combinations thereof. Anthelminties may be administered by a variety of suitable routes, including orally, parenterally, such as subcutaneously, intravenously, intramuscularly or interperitoneally, or topically (cutaneously), such as directly on to exposed skin surface, to a patient in the treatment and/or prevention of intestinal parasites.

Therapeutics for the treatment of Giardia infections are widely known to those skilled in the art. Giardia infections may be treated with a one or more of several drugs including fenbendazole, albendazole, metronidazole, tinidazole, nitazoxanide, paromomycin, quinacrine, and furazolidone, febantel, pyrantel pamoate, praziquantel or combinations thereof. These drugs may be administered by a variety of suitable routes, including orally, parenterally, such as subcutaneously, intravenously, intramuscularly or interperitoneally, or topically (cutaneously), such as directly on to exposed skin surface, to a patient in the treatment and/or prevention of Giardia.

Therapeutics for the treatment of Cystoisospora infections are widely known to those skilled in the art. Cystoisospora infections may be treated with a one or more of several drugs including sulfa-type antibiotics, such as sulfadimethoxine or trimethoprim-sulfonamide, and triazine antiprotozoals, including diclazuril, ponazuril and toltrazuril. In cases of severe diarrhea and dehydration, the infected animal may be supported with fluid therapy and nutritional support. Additional drugs for the treatment of Cystoisospora infections include famprolium, ponazuril or toltrazuril, either alone or in combination with sulfa-type antibiotics. The risk of reinfection may be reduced by removing feces of an infected dog as soon as possible Other methods of reducing the risk of reinfection include disinfecting surfaces with treatments that decrease or eliminate the viability of Cystoisospora oocysts. Such surface treatments include diluted chlorine bleach and steam cleaning.

Intermediate hosts may be involved in transmitting one or more worm or non-worm parasites, fungi, viruses and bacteria to the patient and thus a successful therapeutic intervention includes strategies to control or prevent reinfection. For instance, as tapeworm infections can be transmitted by intermediate hosts such as fleas and canine chewing lice, a successful therapeutic intervention in the case of tapeworm infection includes strategies to control any intermediate host (e.g., flea) infestation that may be present on the patient Thus, controlling intermediate hosts such as fleas aids in preventing reinfection of the patient with tapeworm. Therapeutics for the treatment or control of flea infestation are well known to those skill in the art and include selamectin, fipronil, imidacloprid, indoxacarb, pyrethrin, permethrin, flumethrin, spinosad, nitenpyram, afoxolaner, fluralaner, saralane, nitenpyram, methoprene, pyriproxyfen, and lufenuron, or combinations thereof. Flea control agents may be administered by a variety of suitable routes including orally, parenterally, such as subcutaneously, intravenously, intramuscularly or interperitoneally, or topically (cutaneously), such as directly on to exposed skin surface, to a patient. Representative suitable flea control forms includes sprays, powders, collar, oral compositions, or topical treatments.

In some embodiments, kits are provided that are suitable for detecting one or more Dipylidium caninum, Toxocara canis, Ancylostoma canimum, Cystoisospora canis, Trichuris vulpis and/or Giardia duodenalis in an animal host comprising one or more sets of PCR primers and probe(s) specific for a parasite, each primer and probe being complementary to a repetitive species-specific parasite target DNA, and a set of written instructions for detecting the parasite infection. One or more optional swabs may be included in the kit. The swab may be any medical grade, diagnostic grade swab suitable for use in PCR and nucleic acid extraction and may be stored in a provided sleeve or sterile non-additive tube at room temperature or refrigerated or frozen prior to nucleic acid extraction.

Various embodiments of the disclosure are provided below.

Embodiment 1: A method for detecting at least one or more intestinal parasites selected from Dipylidium caninum, Toxocara canis, Ancylostoma canimum, Cystoisospora canis, Trichuris vulpis and Giardia duodenalis from a sample taken from a canine subject, the method comprising:
(a) providing a sample comprising biological material taken from the subject or nucleic acids extracted from the biological material taken from the subject, wherein the sample includes at least 0.1 femtograms of nucleic acids;
(b) providing a composition suitable for detecting the one or more intestinal parasites selected from Dipylidium canimum, Toxocara canis, Ancylostoma canimum, Cystoisospora canis, Trichuris vulpis and Giardia duodenalis, the composition comprising one or more of an oligonucleotide primer pair comprising forward and reverse primers directed to repetitive elements of Dipylidium caninum, Toxocara canis, Ancylostoma canimum, Cystoisospora canis, Trichuris vulpis or Giardia duodenalis;
(c) contacting the sample with the composition of step (b) under conditions suitable for specific hybridization and extension of hybridized nucleic acids by polymerase chain reaction (PCR) to produce one or more PCR extension products, each PCR extension product associated with Dipylidium caninum, Toxocara canis, Ancylostoma canimum, Cystoisospora canis, Trichuris vulpis or Giardia duodenalis;
(d) detecting the one or more PCR extension products as an indication of the presence or absence of one or more intestinal parasites selected from Dipylidium canimum, Toxocara canis, Ancylostoma canimum, Cystoisospora canis, Trichuris vulpis and Giardia duodenalis; and
(e) determining the presence of Dipylidium canimum in the sample if a PCR extension product associated with Dipylidium canimum is detected; the presence of Toxocara canis in the sample if a PCR extension product associated with Toxocara canis is detected; the presence of Ancylostoma caninum in the sample if a PCR extension product associated with Ancylostoma caninum is detected; the presence of Cystoisospora canis in the sample if a PCR extension product associated with Cystoisospora canis is detected, the presence of Trichuris vulpis in the sample if a PCR extension product associated with Trichuris vulpis is detected; and/or the presence of Giardia duodenalis in the sample if a PCR extension product associated with Giardia duodenalis is detected.

Embodiment 2. The method according to Embodiment 1, wherein the sample includes two or more, three or more or four or more intestinal parasites or nucleic acids thereof.

Embodiment 3: The method according to Embodiment 1, wherein the composition further comprises one or more detection probes, each probe specific for the PCR extension product.

Embodiment 4: The method according to Embodiment 3, wherein the detection probe is a hybridization probe or a hydrolysis probe.

Embodiment 5: The method according to Embodiment 3, wherein the one or more detection probes comprises a detectable label selected from the group consisting of radioactive, calorimetric, fluorometric, luminescent and organic labels.

Embodiment 6: The method according to Embodiment 4, wherein the detectable label comprises a fluorometric label.

Embodiment 7: The method according to Embodiment 1, wherein the biological material is collected from the perianal area of the subject.

Embodiment 8: The method according to Embodiment 1, wherein the biological material is collected with a swab.

Embodiment 9: The method according to Embodiment 1, wherein the sample is collected at the point of care.

Embodiment 10: The method according to Embodiment 1, wherein step (c) is carried out at the point of care.

Embodiment 11: The method according to Embodiment 1, wherein the repetitive elements are present in low, medium or high copy number.

Embodiment 12: The method according to Embodiment 1, wherein the repetitive elements are located in at least one of the nuclear genome and the mitochondrial genome.

Embodiment 13: The method according to Embodiment 1, wherein steps (c) and (d) are carried out using one or more oligonucleotide primer/probe sets selected from the group consisting of:

(a)
a 17-mer oligonucleotide forward primer
(SEQ ID NO: 1)
5′ GGCACCTGTCTGTCAGG 3′
and
19-mer reverse primer
(SEQ ID NO: 2)
5′ TCTAAGCGTCTGCAATTCG 3′,
a labeled 27-mer donor probe
(SEQ ID NO: 3)
5′ ACGTTTAATGTTTGCAGAATCGTGACT-FL 3′
and
a labeled 24-mer acceptor probe
(SEQ ID NO: 4)
5′ LC640-ACCTAGCTTCAGCGATGGATCGGT-PH 3′;
(b)
a 17-mer oligonucleotide forward primer
(SEQ ID NO: 5)
5′ TTCCGAACGGCGGATCA 5′
and
16-mer reverse primer
(SEQ ID NO: 6)
5′ CTCAGACAGGCGTAGC 3′,
a labeled 30-mer donor probe
(SEQ ID NO: 7)
5′ TGATGTGAATTGCAGACACACTGAACTTGA-FL 3′
and
a labeled 26-mer acceptor probe
(SEQ ID NO: 8)
5′ LC610-TACTTTGAACGCACATTGCAGCGTCG-PH 3′;
(c)
a 21-mer oligonucleotide forward primer
(SEQ ID NO: 9)
5′ TCTTGCATTTGTGGTTGCCTA 3′
and
22-mer reverse primer
(SEQ ID NO: 10)
5′ GCATCACTCTGAAATACACAAC 3′,
labeled 16-mer donor probe
(SEQ ID NO: 11)
5′ GTTGAGGCCCCCACGA-FL 3′
and
labeled 24-mer acceptor probe
(SEQ ID NO: 12)
5′ LC670-CCAGTATGTTGTTGGCTGGTCTTT-PH 3′;
(d)
a 26-mer oligonucleotide forward primer
(SEQ ID NO: 13)
5′ CTTTTAGAAGATGATTACCTAAGGCT 3′
and
21-mer reverse primer
(SEQ ID NO: 14)
5′ ACGTTCTACTATGAACCAACG 3′,
and
a 20-mer probe
(SEQ ID NO: 15)
5′ LC640-AGTGCGCCGACGCCTGTTAG-BBQ 3′;
(e)
a 17-mer oligonucleotide forward primer
(SEQ ID NO: 16)
5′ GATCCCGTTGTTAGGCA 3′
and
17-mer reverse primer
(SEQ ID NO: 17)
5′ TCGATGACCACACCATG 3′,
and
a labeled 14-mer probe
(SEQ ID NO: 18)
5′ 6-FAM-GTGTGTGCACAGTC-MGB-NFQ 3′;
and
(f)
a 21-mer oligonucleotide forward primer
(SEQ ID NO: 19)
5′ ATTCTCTGCTATTTGGTGACG 3′
and
17-mer reverse primer
(SEQ ID NO: 20)
5′ ACCTTCAGCAACAAGGC 3′,
and
a labeled 26-mer probe
(SEQ ID NO: 21)
5′ LC670-ATGCCCATAGCCAAAGAGAGTCCACG-BBQ 3′.

Embodiment 14: A method for detecting at least one or more intestinal parasites selected from Dipylidium caninum, Toxocara canis, Ancylostoma canimum, Cystoisospora canis, Trichuris vulpis and Giardia duodenalis from a sample taken from a canine subject and treating the canine subject, the method comprising:
(a) providing a sample comprising biological material taken from the subject or nucleic acids extracted from the biological material taken from the subject, wherein the sample includes at least 0.1 femtograms of nucleic acids;
(b) providing a composition suitable for detecting the one or more intestinal parasites selected from Dipylidium canimum, Toxocara canis, Ancylostoma caninum, Cystoisospora canis, Trichuris vulpis and Giardia duodenalis, the composition comprising one or more of an oligonucleotide primer pair comprising forward and reverse primers directed to repetitive elements of Dipylidium caninum, Toxocara canis, Ancylostoma canimum, Cystoisospora canis, Trichuris vulpis or Giardia duodenalis;
(c) Contacting the sample with the composition of step (b) under conditions suitable for specific hybridization and extension of hybridized nucleic acids by polymerase chain reaction (PCR) to produce one or more PCR extension products, each PCR extension product associated with Dipylidium canimum, Toxocara canis, Ancylostoma canimum, Cystoisospora canis, Trichuris vulpis or Giardia duodenalis;
(d) detecting for the one or more PCR extension products as an indication of the presence or absence of one or more intestinal parasites selected from Dipylidium caninum, Toxocara canis, Ancylostoma canimum, Cystoisospora canis, Trichuris vulpis and Giardia duodenalis; and
(e) determining the presence of Dipylidium canimum in the sample if a PCR extension product associated with Dipylidium caninum is detected; the presence of Toxocara canis in the sample if a PCR extension product associated with Toxocara canis is detected; the presence of Ancylostoma caninum in the sample if a PCR extension product associated with Ancylostoma caninum is detected; the presence of Cystoisospora canis in the sample if a PCR extension product associated with Cystoisospora canis is detected; the presence of Trichuris vulpis in the sample if a PCR extension product associated with Trichuris vulpis is detected; and/or the presence of Giardia duodenalis in the sample if a PCR extension product associated with Giardia duodenalis is detected, and
(f) if the presence of one or more Dipylidium canimum, Toxocara canis, Ancylostoma canimum, Cystoisospora canis, Trichuris vulpis and/or Giardia duodenalis is detected, administering to the canine subject a treatment effective at reducing or eliminating parasite infection.

Embodiment 15. The method according to Embodiment 14, wherein the sample includes two or more, three or more or four or more intestinal parasites or nucleic acids thereof.

Embodiment 16: The method according to Embodiment 14, wherein the composition further comprises one or more detection probes, each probe specific for the PCR extension product.

Embodiment 17: The method according to Embodiment 16, wherein the detection probe is a hybridization probe or a hydrolysis probe.

Embodiment 18: The method according to Embodiment 16, wherein the one or more detection probes comprises a detectable label selected from the group consisting of radioactive, calorimetric, fluorometric, luminescent and organic labels.

Embodiment 19: The method according to Embodiment 17, wherein the detectable label comprises a fluorometric label.

Embodiment 20: The method according to Embodiment 14, wherein the biological material is collected from the perianal area of the subject.

Embodiment 21: The method according to Embodiment 14, wherein the biological material is collected with a swab.

Embodiment 22: The method according to Embodiment 14, wherein the sample is collected at the point of care.

Embodiment 23: The method according to Embodiment 14, wherein step (c) is carried out at the point of care.

Embodiment 24: The method according to Embodiment 14, wherein the repetitive elements are present in low, medium or high copy number.

Embodiment 25: The method according to Embodiment 14, wherein the repetitive elements are located in at least one of the nuclear genome and the mitochondrial genome.

Embodiment 26: The method according to Embodiment 14, wherein steps (c) and (d) are carried out using one or more oligonucleotide primer/probe sets selected from the group consisting of:

(a)
a 17-mer oligonucleotide forward primer
(SEQ ID NO: 1)
5′ GGCACCTGTCTGTCAGG 3′
and
19-mer reverse primer
(SEQ ID NO: 2)
5′ TCTAAGCGTCTGCAATTCG 3′,
a labeled 27-mer donor probe
(SEQ ID NO: 3)
5′ ACGTTTAATGTTTGCAGAATCGTGACT-FL 3′
and
a labeled 24-mer acceptor probe
(SEQ ID NO: 4)
5′ LC640-ACCTAGCTTCAGCGATGGATCGGT-PH 3′;
(b)
a 17-mer oligonucleotide forward primer
(SEQ ID NO: 5)
5′ TTCCGAACGGCGGATCA 5′
and
16-mer reverse primer
(SEQ ID NO: 6)
5′ CTCAGACAGGCGTAGC 3′,
a labeled 30-mer donor probe
(SEQ ID NO: 7)
5′ TGATGTGAATTGCAGACACACTGAACTTGA-FL 3′
and
a labeled 26-mer acceptor probe
(SEQ ID NO: 8)
5′ LC610-TACTTTGAACGCACATTGCAGCGTCG-PH 3′;
(c)
a 21-mer oligonucleotide forward primer
(SEQ ID NO: 9)
5′ TCTTGCATTTGTGGTTGCCTA 3′
and
22-mer reverse primer
(SEQ ID NO: 10)
5′ GCATCACTCTGAAATACACAAC 3′,
labeled 16-mer donor probe
(SEQ ID NO: 11)
5′ GTTGAGGCCCCCACGA-FL 3′
and
labeled 24-mer acceptor probe
(SEQ ID NO: 12)
5′ LC670-CCAGTATGTTGTTGGCTGGTCTTT-PH 3′;
(d)
a 26-mer oligonucleotide forward primer
(SEQ ID NO: 13)
5′ CTTTTAGAAGATGATTACCTAAGGCT 3′
and
21-mer reverse primer
(SEQ ID NO: 14)
5′ ACGTTCTACTATGAACCAACG 3′,
and
a 20-mer probe
(SEQ ID NO: 15)
5′ LC640-AGTGCGCCGACGCCTGTTAG-BBQ 3′;
(e)
a 17-mer oligonucleotide forward primer
(SEQ ID NO: 16)
5′ GATCCCGTTGTTAGGCA 3′
and
17-mer reverse primer
(SEQ ID NO: 17)
5′ TCGATGACCACACCATG 3′,
and
a labeled 14-mer probe
(SEQ ID NO: 18)
5′ 6-FAM-GTGTGTGCACAGTC-MGB-NFQ 3′;
and
(f)
a 21-mer oligonucleotide forward primer
(SEQ ID NO: 19)
5′ ATTCTCTGCTATTTGGTGACG 3′
and
17-mer reverse primer
(SEQ ID NO: 20)
5′ ACCTTCAGCAACAAGGC 3′,
and
a labeled 26-mer probe
(SEQ ID NO: 21)
5′ LC670-ATGCCCATAGCCAAAGAGAGTCCACG-BBQ 3′.

Embodiment 27: A kit for detecting one or more intestinal parasites selected from Dipylidium canimum, Toxocara canis, Ancylostoma caninum, Cystoisospora canis, Trichuris vulpis and Giardia duodenalis from a sample taken from a canine subject, the kit comprising:
(a) one or more oligonucleotide primer pairs comprising of forward and reverse primers directed to repetitive elements of Dipylidium caninum, Toxocara canis, Ancylostoma caninum, Cystoisospora canis, Trichuris vulpis or Giardia duodenalis;
(b) detection probes for detecting PCR extension products associated with the one or more intestinal parasites selected from Dipylidium caninum, Toxocara canis, Ancylostoma caninum, Cystoisospora canis, Trichuris vulpis and Giardia duodenalis; and
(c) optional buffers and a set of instructions.

Embodiment 28: The kit according to Embodiment 27, wherein parts (i) and (ii) are part of an oligonucleotide primer/probe set, the oligonucleotide primer/probe set selected from the group consisting of:

(a)
a 17-mer oligonucleotide forward primer
(SEQ ID NO: 1)
5′ GGCACCTGTCTGTCAGG 3′
and
19-mer reverse primer
(SEQ ID NO: 2)
5′ TCTAAGCGTCTGCAATTCG 3′,
a labeled 27-mer donor probe
(SEQ ID NO: 3)
5′ ACGTTTAATGTTTGCAGAATCGTGACT-FL 3′
and
a labeled 24-mer acceptor probe
(SEQ ID NO: 4)
5′ LC640-ACCTAGCTTCAGCGATGGATCGGT-PH 3′;
(b)
a 17-mer oligonucleotide forward primer
(SEQ ID NO: 5)
5′ TTCCGAACGGCGGATCA 5′
and
16-mer reverse primer
(SEQ ID NO: 6)
5′ CTCAGACAGGCGTAGC 3′,
a labeled 30-mer donor probe
(SEQ ID NO: 7)
5′ TGATGTGAATTGCAGACACACTGAACTTGA-FL 3′
and
a labeled 26-mer acceptor probe
(SEQ ID NO: 8)
5′ LC610-TACTTTGAACGCACATTGCAGCGTCG-PH 3′;
(c)
a 21-mer oligonucleotide forward primer
(SEQ ID NO: 9)
5′ TCTTGCATTTGTGGTTGCCTA 3′
and
22-mer reverse primer
(SEQ ID NO: 10)
5′ GCATCACTCTGAAATACACAAC 3′,
labeled 16-mer donor probe
(SEQ ID NO: 11)
5′ GTTGAGGCCCCCACGA-FL 3′
and
labeled 24-mer acceptor probe
(SEQ ID NO: 12)
5′ LC670-CCAGTATGTTGTTGGCTGGTCTTT-PH 3′;
(d)
a 26-mer oligonucleotide forward primer
(SEQ ID NO: 13)
5′ CTTTTAGAAGATGATTACCTAAGGCT 3′
and
21-mer reverse primer
(SEQ ID NO: 14)
5′ ACGTTCTACTATGAACCAACG 3′,
and
a 20-mer probe
(SEQ ID NO: 15)
5′ LC640-AGTGCGCCGACGCCTGTTAG-BBQ 3′;
(e)
a 17-mer oligonucleotide forward primer
(SEQ ID NO: 16)
5′ GATCCCGTTGTTAGGCA 3′
and
17-mer reverse primer
(SEQ ID NO: 17)
5′ TCGATGACCACACCATG 3′,
and
a labeled 14-mer probe
(SEQ ID NO: 18)
5′ 6-FAM-GTGTGTGCACAGTC-MGB-NFQ 3′;
and
(f)
a 21-mer oligonucleotide forward primer
(SEQ ID NO: 19)
5′ ATTCTCTGCTATTTGGTGACG 3′
and
17-mer reverse primer
(SEQ ID NO: 20)
5′ ACCTTCAGCAACAAGGC 3′,
and
a labeled 26-mer probe
(SEQ ID NO: 21)
5′ LC670-ATGCCCATAGCCAAAGAGAGTCCACG-BBQ 3′.

Embodiment 29: A oligonucleotide primer/probe set selected from the group consisting of: a 17-mer oligonucleotide forward primer 5′ GGCACCTGTCTGTCAGG 3′ (SEQ ID NO: 1) and 19-mer reverse primer 5′ TCTAAGCGTCTGCAATTCG 3′ (SEQ ID NO: 2), a labeled 27-mer donor probe 5′ ACGTTTAATGTTTGCAGAATCGTGACT-FL 3′ (SEQ ID NO: 3) and a labeled 24-mer acceptor probe 5′ LC640-ACCTAGCTTCAGCGATGGATCGGT-PH 3′ (SEQ ID NO: 4);

(a)
a 17-mer oligonucleotide forward primer
(SEQ ID NO: 1)
5′ GGCACCTGTCTGTCAGG 3′
and
19-mer reverse primer
(SEQ ID NO: 2)
5′ TCTAAGCGTCTGCAATTCG 3′,
a labeled 27-mer donor probe
(SEQ ID NO: 3)
5′ ACGTTTAATGTTTGCAGAATCGTGACT-FL 3′
and
a labeled 24-mer acceptor probe
(SEQ ID NO: 4)
5′ LC640-ACCTAGCTTCAGCGATGGATCGGT-PH 3′;
(b)
a 17-mer oligonucleotide forward primer
(SEQ ID NO: 5)
5′ TTCCGAACGGCGGATCA 5′
and
16-mer reverse primer
(SEQ ID NO: 6)
5′ CTCAGACAGGCGTAGC 3′,
a labeled 30-mer donor probe
(SEQ ID NO: 7)
5′ TGATGTGAATTGCAGACACACTGAACTTGA-FL 3′
and
a labeled 26-mer acceptor probe
(SEQ ID NO: 8)
5′ LC610-TACTTTGAACGCACATTGCAGCGTCG-PH 3′;
(c)
a 21-mer oligonucleotide forward primer
(SEQ ID NO: 9)
5′ TCTTGCATTTGTGGTTGCCTA 3′
and
22-mer reverse primer
(SEQ ID NO: 10)
5′ GCATCACTCTGAAATACACAAC 3′,
labeled 16-mer donor probe
(SEQ ID NO: 11)
5′ GTTGAGGCCCCCACGA-FL 3′
and
labeled 24-mer acceptor probe
(SEQ ID NO: 12)
5′ LC670-CCAGTATGTTGTTGGCTGGTCTTT-PH 3′;
(d)
a 26-mer oligonucleotide forward primer
(SEQ ID NO: 13)
5′ CTTTTAGAAGATGATTACCTAAGGCT 3′
and
21-mer reverse primer
(SEQ ID NO: 14)
5′ ACGTTCTACTATGAACCAACG 3′,
and
a 20-mer probe
(SEQ ID NO: 15)
5′ LC640-AGTGCGCCGACGCCTGTTAG-BBQ 3′;
(e)
a 17-mer oligonucleotide forward primer
(SEQ ID NO: 16)
5′ GATCCCGTTGTTAGGCA 3′
and
17-mer reverse primer
(SEQ ID NO: 17)
5′ TCGATGACCACACCATG 3′,
and
a labeled 14-mer probe
(SEQ ID NO: 18)
5′ 6-FAM-GTGTGTGCACAGTC-MGB-NFQ 3′;
and
(f)
a 21-mer oligonucleotide forward primer
(SEQ ID NO: 19)
5′ ATTCTCTGCTATTTGGTGACG 3′
and
17-mer reverse primer
(SEQ ID NO: 20)
5′ ACCTTCAGCAACAAGGC 3′,
and
a labeled 26-mer probe
(SEQ ID NO: 21)
5′ LC670-ATGCCCATAGCCAAAGAGAGTCCACG-BBQ 3′.

### Example A

Unless otherwise indicated, the following materials and techniques were used to generate data described in one or more of Examples 1˜4 as described below.

### Origin of Samples and Sample Preparation

Canine perianal swabs were collected from shelter dogs and pooled. All dogs were negative for A. caninum, Trichuris vulpis and T. canis as determined by fecal antigen immunoassays and by centrifugal fecal flotation (flotation is also referred to as O&P orOva and Parasites). Fecal antigen immunoassay were performed as described in U.S. Pat. No. 10,942,180. Centrifugal fecal flotation was performed according to the zinc sulfate method as described in Zajac. (2013). Veterinary clinical parasitology, eighth edition. Wiley-Blackwell.

### Genomic DNA Extraction Procedure

Canine swab DNA was extracted from perianal swabs using the High Pure PCR Template Preparation Kit (Roche Diagnostics; Catalog #: 11796828001) according to the manufacturer's instructions.

### Specific Primer Design

A. Primer/Probe Designs with Hybridization Probes (FRET):

All donor and acceptor probes were purchased with labels and/or caps from the vendors noted below. All donor probes were 3′ labeled with FITC (fluorescein isothiocyanate; noted as “FL” in the sequences). Acceptor probes had a 3′ cap (noted as “PH” in the sequences).

The acceptor probe for Ancylostoma caninum was 5′ labeled with LightCycler® Red 640 (noted as “LC640” in the sequences below) (purchased from Integrated DNA Technologies, Coralville, Iowa, USA).

The acceptor probe for Trichuris vulpis was 5′ labeled with LightCycler® Red 610 (noted as “LC610” in the sequences below) (purchased from Integrated DNA Technologies, Coralville, Iowa, USA).

The acceptor probe for Toxocara canis was 5′ labeled with LC® Red 670 (noted as “LC670” in the sequences below) (purchased from Integrated DNA Technologies, Coralville, Iowa, USA).

Design for the amplification and detection of ITS1-5.8S rDNA of Ancylostoma caninum (the “A. c. rDNA Primer/Probe Set”)

Forward primer: 17 mer
(SEQ ID NO: 1)
5′ GGCACCTGTCTGTCAGG 3′
Reverse primer: 19 mer
(SEQ ID NO: 2)
5′ TCTAAGCGTCTGCAATTCG 3′
Donor probe: 27 mer
(SEQ ID NO: 3)
5′ ACGTTTAATGTTTGCAGAATCGTGACT-FITC 3′
Acceptor probe: 24 mer
(SEQ ID NO: 4)
5′ LC640-ACCTAGCTTCAGCGATGGATCGGT-PH 3′

Design for the amplification and detection of 5.8S rDNA of Trichuris vulpis (the “Trich. rDNA Primer/Probe Set”)

Forward primer: 17 mer
(SEQ ID NO: 5)
5′ TTCCGAACGGCGGATCA 5′
Reverse primer: 16 mer
(SEQ ID NO: 6)
5′ CTCAGACAGGCGTAGC 3′
Donor probe: 30 mer
(SEQ ID NO: 7)
5′ TGATGTGAATTGCAGACACACTGAACTTGA-FITC 3′
Acceptor probe: 26 mer
(SEQ ID NO: 8)
5′ LC610-TACTTTGAACGCACATTGCAGCGTCG-PH 3′

Design for the amplification and detection of the mitochondrial NADH subunit 4 of Toxocara canis (the “T. c. NADH Primer/Probe Set”):

Forward primer: 21 mer
(SEQ ID NO: 9)
5′ TCTTGCATTTGTGGTTGCCTA 3′
Reverse primer: 22 mer
(SEQ ID NO: 10)
5′ GCATCACTCTGAAATACACAAC 3′
Donor probe: 16 mer
(SEQ ID NO: 11)
5′ GTTGAGGCCCCCACGA-FITC 3′
Acceptor probe: 24 mer
(SEQ ID NO: 12)
5′ LC670-CCAGTATGTTGTTGGCTGGTCTTT-PH 3′

B. Primer/Probe Designs with Hydrolysis Probes:

Probes were conjugated to fluorescent labels and quenchers as noted in the sequences below (LC640 indicates LC® Red 640; LC670 indicates LC® Red 670; FAM indicates 6-carboxyfluorescein; NFQ-MGB indicates Nonfluorescent Quencher-Minor Groove Binder).

Oligonucleotides conjugated to a BlackBerry™ Quencher (BBQ) were purchased from LGC Biosearch Technologies (Novato, California, USA).

Design for the amplification and detection of the mitochondrial NADH subunit 5 of Ancylostoma caninum (the “A. c. NADH Primer/Probe Set”)

Forward primer: 26 mer
(SEQ ID NO: 13)
5′ CTTTTAGAAGATGATTACCTAAGGCT 3′
Reverse primer: 21 mer
(SEQ ID NO: 14)
5′ ACGTTCTACTATGAACCAACG 3′
Probe: 20 mer
(SEQ ID NO: 15)
5′ LC640-AGTGCGCCGACGCCTGTTAG-BBQ 3′

Design for the amplification and detection of 28S rDNA of Dipylidium caninum (the “D. c. rDNA Primer/Probe Set”)

Forward primer: 17 mer
(SEQ ID NO: 16)
5′ GATCCCGTTGTTAGGCA 3′
Reverse primer: 17 mer
(SEQ ID NO: 17)
5′ TCGATGACCACACCATG 3′
Probe: 14 mer
(SEQ ID NO: 18)
5′ 6-FAM-GTGTGTGCACAGTC-MGB-NFQ 3′

Design for the amplification and detection of ITS1 rDNA of Cystoisospora canis (the “C. c. rDNA Primer/Probe Set”)

Forward primer: 21 mer
(SEQ ID NO: 19)
5′ ATTCTCTGCTATTTGGTGACG 3′
Reverse primer: 17 mer
(SEQ ID NO: 20)
5′ ACCTTCAGCAACAAGGC 3′
Probe: 26 mer
(SEQ ID NO: 21)
5′ LC670-ATGCCCATAGCCAAAGAGAGTCCACG-BBQ 3′

### Specific Target Design

A. c. rDNA Target Plasmid

A plasmid containing sequences from the ITS1-5.8S rDNA of Ancylostoma caninum (the “A. c. rDNA Target Plasmid”) was constructed to encompass the region defined by the A. c. rDNA Primer/Probe Set.

Trich. rDNA Target Plasmid

A plasmid containing sequences from the 5.8S rDNA of Trichuris vulpis (the “Trich. rDNA Target Plasmid”) was constructed to encompass the region defined by the Trich. rDNA Primer/Probe Set.

T. c. NADH Target Plasmid

A plasmid containing sequences from the mitochondrial NADH subunit 4 of Toxocara canis (the “T. c. NADH Target Plasmid”) was constructed to encompass the region defined by the T. c. NADH Primer/Probe Set.

A. c. NADH Target Plasmid

A plasmid containing sequences from the mitochondrial NADH subunit 5 of Ancylostoma caninum (the “A. c. NADH Target Plasmid”) was constructed to encompass the region defined by the A. c. NADH Primer/Probe Set.

D. c. rDNA Target Plasmid

A plasmid containing sequences from the 28S rDNA of Dipylidium canimum (the “D. c. rDNA Target Plasmid”) was constructed to encompass region defined by the D. c. rDNA Primer/Probe Set.

C. c. rDNA Target Plasmid

A plasmid containing sequences from the ITS1 rDNA of Cystoisospora canis (the “C. c. rDNA Target Plasmid”) was constructed to encompass region defined by the C. c. rDNA Primer/Probe Set.

### Master Mix Preparation

The LightCycler® 480 Genotyping Master (Roche; Catalog #04707524001) (“Genotyping Master”) was used as the master mix for all assays using hybridization probes. The LightCycler® 480 Probes Master (Roche; Catalog #04707494001) (“Probes Master”) was used as the master mix for all assays using hydrolysis probes.

### Thermocycling

Thermocycling was performed on a LightCycler® 480 Instrument II (Roche; Catalog #05015243001) using the following cycling programs:
A. Assay designs that include hybridization probes were amplified using the Hybridization Probes Program:

| | |
|---|---|
| Activation (1 cycle) | 95° C. for 10 minutes (required to activate |
| | the polymerase) |
| Amplification | (50 cycles) 95° C. for 20 seconds |
| | 60° C. for 30 seconds (single signal |
| | acquisition) |
| | 72° C. for 20 seconds |
| Melt Curve (1 cycle) | 95° C. for 1 minute |
| | 45° C. for 1 minute |
| | 80° C. continuous temperature increase at |
| | rate of 0.04° C./s |
| | With 4 signal acquisitions per ° C. |
| Cooling (1 cycle) | 45° C. for 30 seconds |

B. Assay designs that include hydrolysis probes were amplified using the Hydrolysis Probes Program:

| | |
|---|---|
| Activation (1 cycle) | 95° C. for 10 minutes (required to activate |
| | the polymerase) |
| Amplification (45 cycles) | 95° C. for 10 seconds |
| | 60° C. for 30 seconds |
| | 72° C. for 1 second (single signal |
| | acquisition) |
| Cooling (1 cycle) | 40° C. for 30 seconds |

### Single PCR Procedure

Specific PCRs were initially tested for each pair of primers described above.

For assays with hybridization probes, the reactions were set up with each Primer/Probe Set, and a series of tenfold dilutions of each Target Plasmid (10 pg to 0.1 fg) or no plasmid (nc, negative control)) and Genotyping Master, and run in triplicate with the Hybridization Probes Program on a LightCycler instrument. Primers were used at a concentration of 0.3 μM for the forward primer and 0.6 μM for the reverse primer. Both probes were used at a concentration of 0.3 μM in a final reaction volume of 20 uL which contained 5 ul of plasmid dilution.

For assays with hydrolysis probes, the reactions were set up with with each Primer/Probe Set, and a series of tenfold dilutions of each Target Plasmid (10 pg to 0.1 fg) or no plasmid (nc, negative control)) and Probes Master, and run in triplicate with the Hydrolysis Probes Program on a LightCycler instrument.

### Multiplex PCR Procedure

Based on the results of the single PCR reactions, multiplex PCR reactions were then carried out.

### Identification of PCR Extension Products

PCR extension products were detected by real-time PCR on a LightCycler® instrument. In the hybridization assays, extension products were detected by an increase of emission wavelength intensity of the acceptor fluorophore caused by FRET between the donor and acceptor probes. In the hydrolysis assays, extension products were detected as an increase in the emission wavelength intensity from the fluorophores caused by separation from the quenchers through hydrolysis of the probes. The cycle number at which the signal first rises above background (and thus indicates detection of the target sequence) is the crossing point (CP).

### Example 1: Single PCR Assays Using Hybridization Detection

In this Example, individual PCR assays were carried out to evaluate the performance of low/moderately repetitive copy target ribosomal genes as well as high copy target mitochondrial genes.

Each of the PCR assays using hybridization probes was run individually in the presence of primers and probes of the two other hybridization probe assays in order to determine the limit of detection, and to assess any interference between the three primer/probe sets.

To this end, PCR reactions for A. c. were set up with the A. c. rDNA Target Plasmid, and the A. c. rDNA Primer/Probe Set, as well as the Trich. rDNA Primer/Probe Set and the T. c. NADH Primer/Probe Set.

Similarly, PCR reactions for Trich. were set up with the Trich. rDNA Target Plasmid, and the Trich. rDNA Primer/Probe Set, as well as the A.c. rDNA Primer/Probe Set and the T. c. NADH Primer/Probe Set.

Further, PCR reactions for T. c. were set up with the T. c. NADH Target Plasmid, and the T. c. NADH Primer/Probe Set, as well as the A.c. rDNA Primer/Probe Set and the Trich. rDNA Primer/Probe Set.

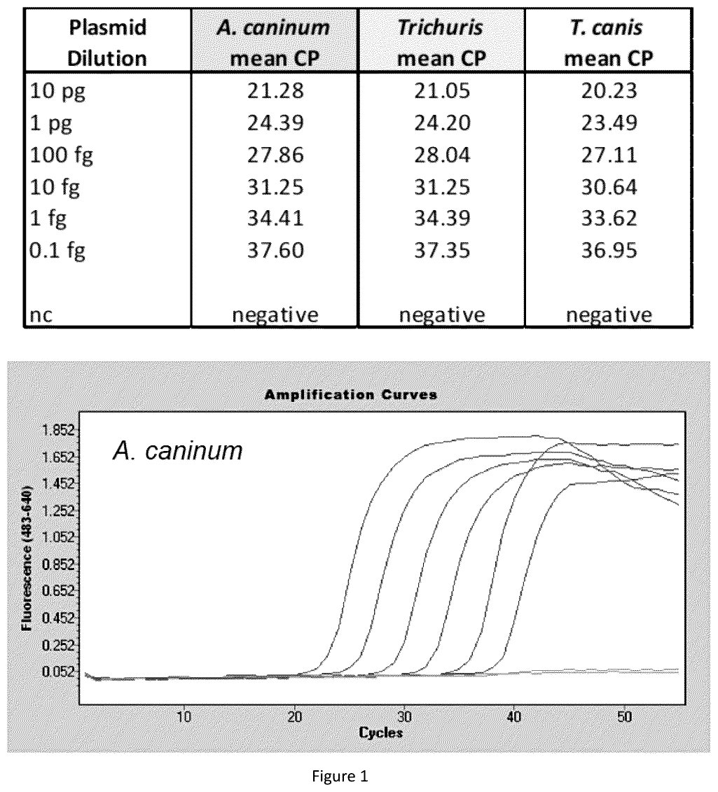

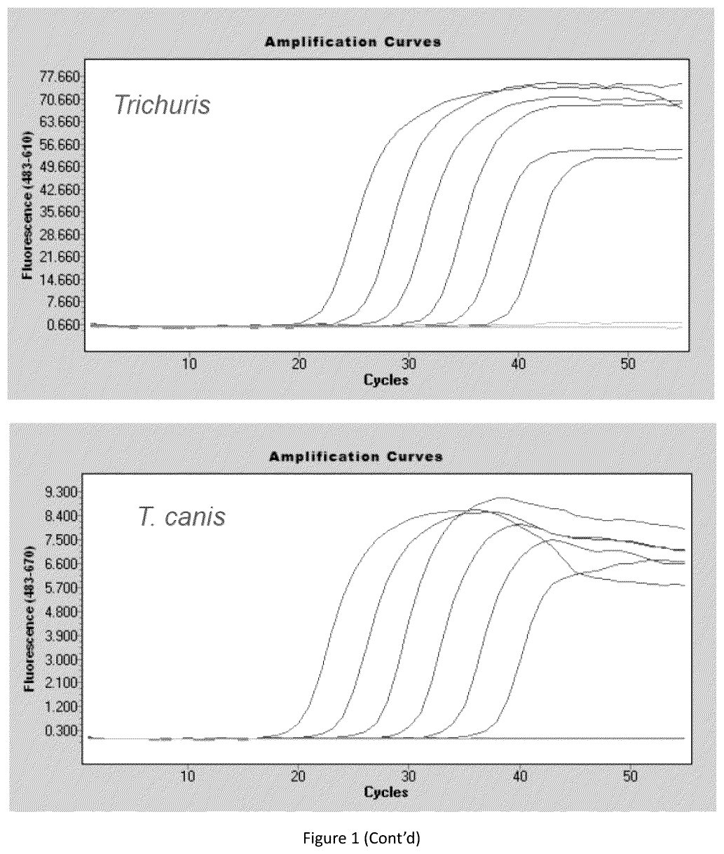

The results are shown in FIG. 1. The table in FIG. 1 shows the mean crossing points (CP) for each assay at each plasmid amount. Each assay was able to detect its Target Plasmid at a plasmid amount as low as 0.1 fg. The negative control is indicated by “ng” (i.e., no Target Plasmid). Therefore, the limit of detection was at least as low as 0.1 fg of each plasmid. The CP values did not indicate that combining the three primer/probe sets affected the limit of detection.

### Example 2: Multiplex Real Time PCR Assays Using Hybridization Detection Probes Sensitivity not Affected by Multiple Targets and Background Sample DNA

In this Example, multiplex PCR assays were carried out to verify and evaluate the performance of a sample including A. canimum (low copy target), T. canis (high copy target), and Trichuris vulpis (low copy target) using hybridization detection probes.

All three PCR assays using hybridization probes were run in multiplex (i.e., all three in the same reaction), in the presence of all three Target Plasmids, and in the presence of canine swab DNA. Primers were used at a concentration of 0.3 μM for the forward primer and 0.6 μM for the reverse primer. Both probes were used at a concentration of 0.3 μM in a final reaction volume of 20 uL which contained 5 ul of nucleic acid extract and 5 uL of plasmid dilution.

Hence, the reaction mix comprised canine swab DNA, A. c. rDNA Target Plasmid, Trich. rDNA Target Plasmid, T. c. NADH Target Plasmid, and the A. c. rDNA Primer/Probe Set, Trich. rDNA Primer/Probe Set and T. c. NADH Primer/Probe Set.

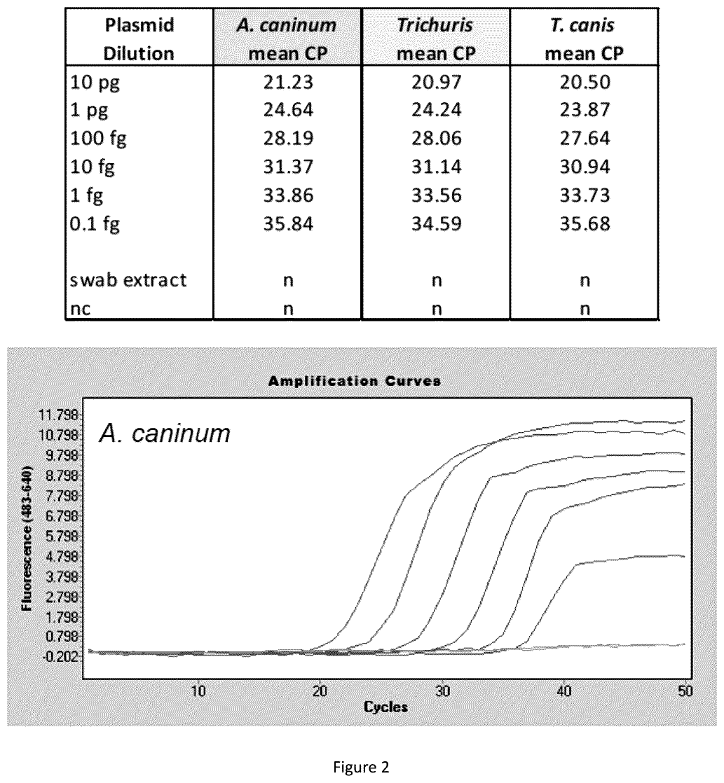

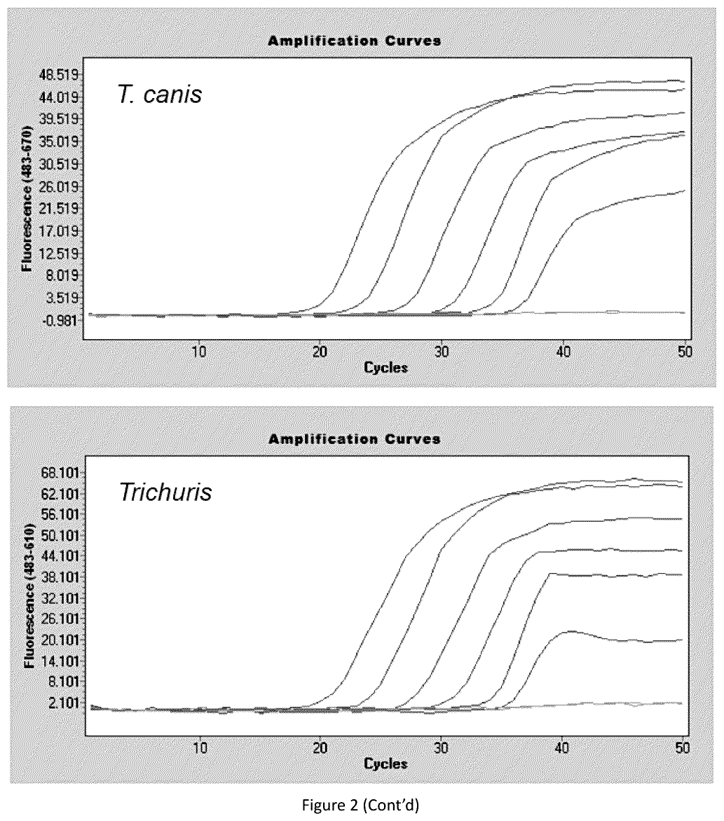

The results are shown in FIG. 2. The table in FIG. 2 shows the mean crossing points (CP) for each assay at each plasmid amount. All samples contained canine swab DNA. The negative control indicated by “swab extract” did not contain any Target Plasmid. The negative control indicated by “nc” contained neither Target Plasmid nor canine swab DNA. Each assay was able to detect its Target Plasmid at a plasmid amount as low as 0.1 fg in the presence of canine swab DNA. Therefore, the limit of detection was at least as low as 0.1 fg of each plasmid in the presence of canine swab DNA.

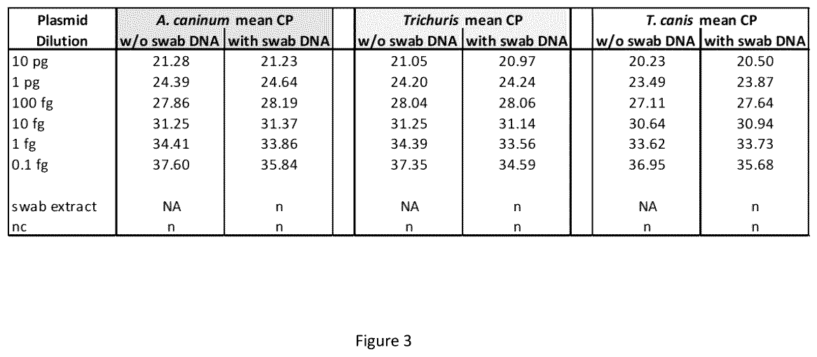

FIG. 3 shows a side-by side comparison of the CP values shown in FIG. 1 and FIG. 2. In each case, the CP values obtained by amplification in the presence of canine swab DNA closely track the CP values obtained by amplification in the absence of canine swab DNA. Therefore, assay sensitivity was not affected by the presence of multiple targets and background sample DNA.

### Example 3: Single Real Time PCR Assays Using Hydrolysis Detection Probes

In this Example, individual PCR assays were carried out to evaluate the performance of PCR reactions using hydrolysis probes for the amplification of low/moderately repetitive copy target ribosomal genes as well as high copy target mitochondrial genes.

Each of the PCR assays using hydrolysis probes was run individually in the presence of primers and probes of the two other hydrolysis probe assays in order to determine the limit of detection, and to assess any interference between the three primer/probe sets. To this end, PCR reactions for A. c. were set up with the A. c. NADH Target Plasmid and the A. c. NADH Primer/Probe Set, as well as the D. c. rDNA Primer/Probe Set and the C. c. rDNA Primer/Probe Set. Primers were used at a final concentration of 0.83 μM and the probe was used at a final concentration of 0.16 μM. The primers and probe were added to the 1× Probes Master mix in a 20-μL final volume to which 5 μL of plasmid dilution was added.

Similarly, PCR reactions for D.c. were set up with the D. c. rDNA Target Plasmid and the D. c. rDNA Primer/Probe Set, as well as the A. c. NADH Primer/Probe Set, and the C. c. rDNA Primer/Probe Set.

Further, PCR reactions for C.c. were set up with the C. c. rDNA Target Plasmid and the C. c. rDNA Primer/Probe Set, as well as the D. c. rDNA Primer/Probe Set, and the A. c. NADH Primer/Probe Set.

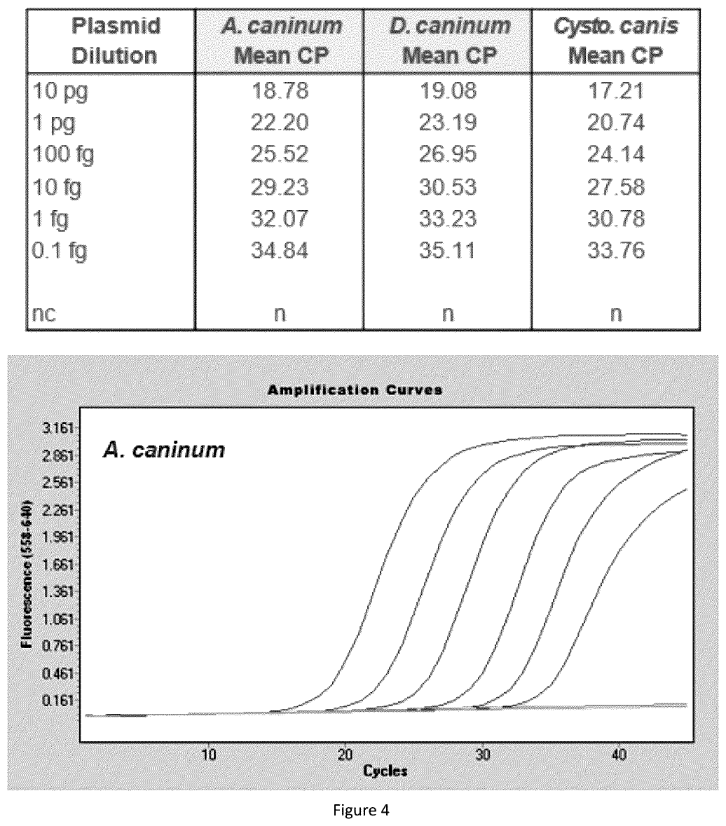

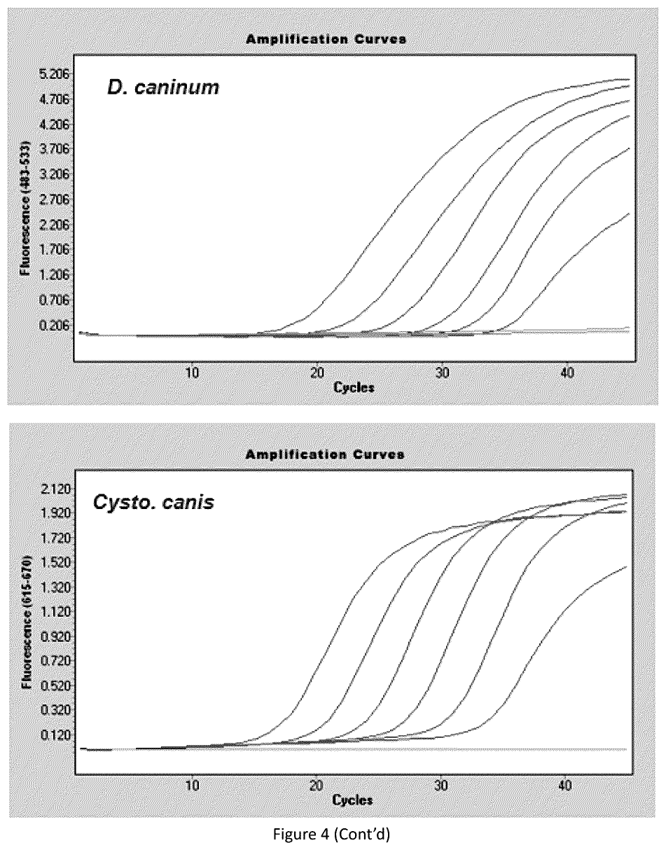

The results are shown in FIG. 4. The table in FIG. 4 shows the mean crossing points (CP) for each assay at each plasmid amount. Each assay was able to detect its Target Plasmid at a plasmid amount as low as 0.1 femtogram (fg). The negative control is indicated by “nc” (i.e., no Target Plasmid). Therefore, the limit of detection was at least as low as 0.1 fg of each plasmid. The CP values did not indicate that combining the three primer/probe sets affected the limit of detection.

### Example 4: Multiplex Real Time PCR Assays Using Hydrolysis Detection Probes Sensitivity not Affected by Multiple Targets and Background Sample DNA

In this Example, multiplex PCR assays were carried out to verify and evaluate the performance of a sample including Ancylostoma caninum (high copy target), Dipylidium caninum (low copy target), and Cystoisospora canis (low copy target) using hydrolysis detection probes. All three PCR assays using hydrolysis probes were run in multiplex (i.e., all three in the same reaction), in the presence of all three Target Plasmids, and in the presence of canine swab DNA. Primers were used at a final concentration of 0.83 μM and the probe was used at a final concentration of 0.16 μM. The primers and probe were added to the 1× Probes Master mix in a 20-μL final volume which contained 5 μL of nucleic acid extract and 5 μL of plasmid dilution.

Hence, the reaction mix comprised canine swab DNA, A. c. NADH Target Plasmid, D.c. rDNA Target Plasmid, C. c. rDNA Target Plasmid, and the A. c. NADH Primer/Probe Set, D.c. rDNA Primer/Probe Set and C. c. rDNA Primer/Probe Set.

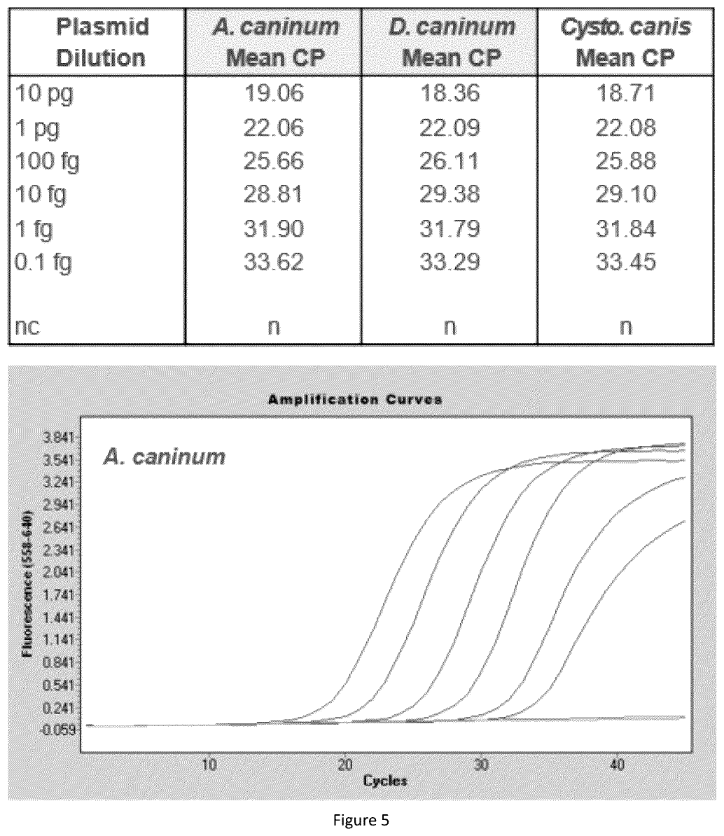

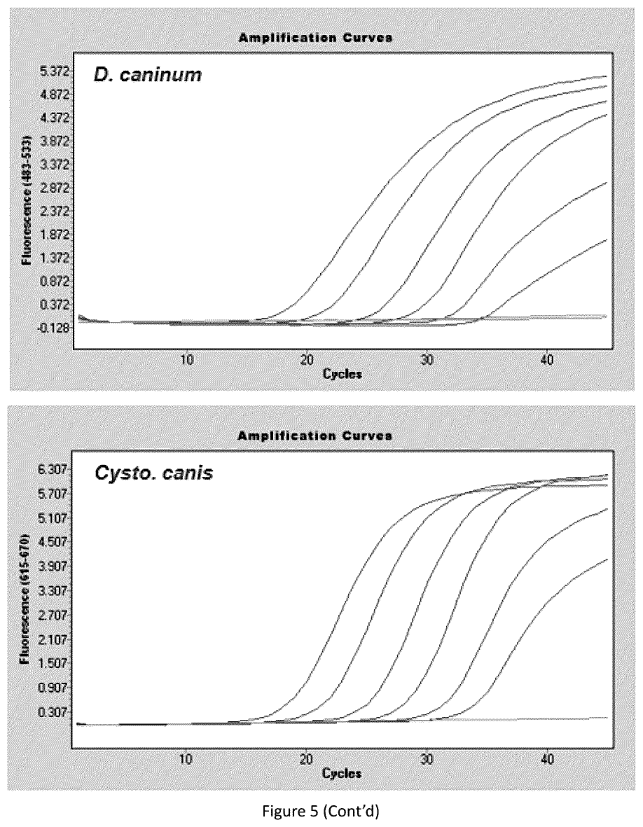

The results are shown in FIG. 5. The table in FIG. 5 shows the mean crossing points (CP) for each assay at each plasmid amount. The negative control indicated by “nc” did not contain any Target Plasmid or canine swab DNA. Each assay was able to detect its Target Plasmid at a plasmid amount as low as 0.1 fg in the presence of canine swab DNA. Therefore, the limit of detection was at least as low as 0.1 fg of each plasmid in the presence of canine swab DNA.

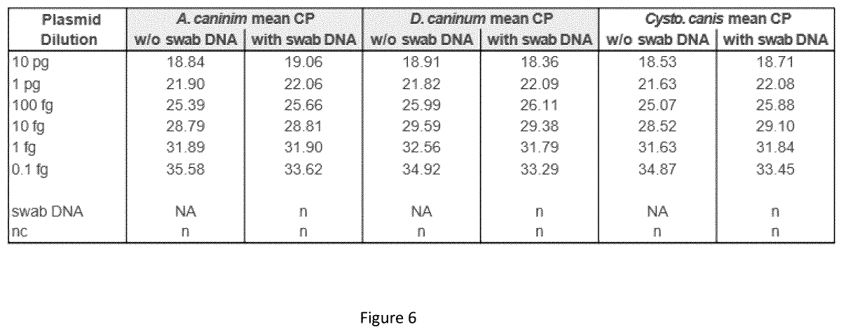

FIG. 6 shows a side-by side comparison of the CP values shown in FIG. 4 and FIG. 5. In each case, the CP values obtained by amplification in the presence of canine swab DNA closely track the CP values obtained by amplification in the absence of canine swab DNA. Therefore, assay sensitivity was not affected by the presence of multiple targets and background sample DNA

What is claimed:

1. A method for detecting at least one or more intestinal parasites selected from Dipylidium caninum, Toxocara canis, Ancylostoma canimum, Cystoisospora canis, Trichuris vulpis and Giardia duodenalis from a sample taken from a canine subject, the method comprising:
(a) providing a sample comprising biological material taken from the subject or nucleic acids extracted from the biological material taken from the subject, wherein the sample includes at least 0.1 femtograms of nucleic acids;
(b) providing a composition suitable for detecting the one or more intestinal parasites selected from Dipylidium caninum, Toxocara canis, Ancylostoma caninum, Cystoisospora canis, Trichuris vulpis and Giardia duodenalis, the composition comprising one or more of an oligonucleotide primer pair comprising forward and reverse primers directed to repetitive elements of Dipylidium caninum, Toxocara canis, Ancylostoma caninum, Cystoisospora canis, Trichuris vulpis or Giardia duodenalis;
(c) contacting the sample with the composition of step (b) under conditions suitable for specific hybridization and extension of hybridized nucleic acids by polymerase chain reaction (PCR) to produce one or more PCR extension products, each PCR extension product associated with Dipylidium caninum, Toxocara canis, Ancylostoma caninum, Cystoisospora canis, Trichuris vulpis or Giardia duodenalis;
(d) detecting the one or more PCR extension products as an indication of the presence or absence of one or more intestinal parasites selected from Dipylidium caninum, Toxocara canis, Ancylostoma caninum, Cystoisospora canis, Trichuris vulpis and Giardia duodenalis; and
(e) determining the presence of Dipylidium canimum in the sample if a PCR extension product associated with Dipylidium canimum is detected; the presence of Toxocara canis in the sample if a PCR extension product associated with Toxocara canis is detected; the presence of Ancylostoma caninum in the sample if a PCR extension product associated with Ancylostoma caninum is detected; the presence of Cystoisospora canis in the sample if a PCR extension product associated with Cystoisospora canis is detected; the presence of Trichuris vulpis in the sample if a PCR extension product associated with Trichuris vulpis is detected; and/or the presence of Giardia duodenalis in the sample if a PCR extension product associated with Giardia duodenalis is detected.

2. The method according to claim 1, wherein the sample includes two or more, three or more or four or more intestinal parasites or nucleic acids thereof.

3. The method according to claim 1, wherein the composition further comprises one or more detection probes, each probe specific for the PCR extension product.

4. The method according to claim 3, wherein the detection probe is a hybridization probe or a hydrolysis probe.

5. The method according to claim 3, wherein the one or more detection probes comprises a detectable label selected from the group consisting of radioactive, calorimetric, fluorometric, luminescent and organic labels.

6. The method according to claim 4, wherein the detectable label comprises a fluorometric label.

7. The method according to claim 1, wherein the biological material is collected from the perianal area of the subject.

8. The method according to claim 1, wherein the biological material is collected with a swab.

9. The method according to claim 1, wherein the sample is collected at the point of care.

10. The method according to claim 1, wherein step (c) is carried out at the point of care.

11. The method according to claim 1, wherein the repetitive elements are present in low, medium or high copy number.

12. The method according to claim 1, wherein the repetitive elements are located in at least one of the nuclear genome and the mitochondrial genome.

13. The method according to claim 1, wherein steps (c) and (d) are carried out using one or more oligonucleotide primer/probe sets selected from the group consisting of:
(a)
a 17-mer oligonucleotide forward primer
(SEQ ID NO: 1)
5′ GGCACCTGTCTGTCAGG 3′
and
19-mer reverse primer
(SEQ ID NO: 2)
5′ TCTAAGCGTCTGCAATTCG 3′,
a labeled 27-mer donor probe
(SEQ ID NO: 3)
5′ ACGTTTAATGTTTGCAGAATCGTGACT-FL 3′
and
a labeled 24-mer acceptor probe
(SEQ ID NO: 4)
5′ LC640-ACCTAGCTTCAGCGATGGATCGGT-PH 3′;
(b)
a 17-mer oligonucleotide forward primer
(SEQ ID NO: 5)
5′ TTCCGAACGGCGGATCA 5′
and
16-mer reverse primer
(SEQ ID NO: 6)
5′ CTCAGACAGGCGTAGC 3′,
a labeled 30-mer donor probe
(SEQ ID NO: 7)
5′ TGATGTGAATTGCAGACACACTGAACTTGA-FL 3′
and
a labeled 26-mer acceptor probe
(SEQ ID NO: 8)
5′ LC610-TACTTTGAACGCACATTGCAGCGTCG-PH 3′;
(c)
a 21-mer oligonucleotide forward primer
(SEQ ID NO: 9)
5′ TCTTGCATTTGTGGTTGCCTA 3′
and
22-mer reverse primer
(SEQ ID NO: 10)
5′ GCATCACTCTGAAATACACAAC 3′,
labeled 16-mer donor probe
(SEQ ID NO: 11)
5′ GTTGAGGCCCCCACGA-FL 3′
and
labeled 24-mer acceptor probe
(SEQ ID NO: 12)
5′ LC670-CCAGTATGTTGTTGGCTGGTCTTT-PH 3′;
(d)
a 26-mer oligonucleotide forward primer
(SEQ ID NO: 13)
5′ CTTTTAGAAGATGATTACCTAAGGCT 3′
and
21-mer reverse primer
(SEQ ID NO: 14)
5′ ACGTTCTACTATGAACCAACG 3′,
and
a 20-mer probe
(SEQ ID NO: 15)
5′ LC640-AGTGCGCCGACGCCTGTTAG-BBQ 3′;
(e)
a 17-mer oligonucleotide forward primer
(SEQ ID NO: 16)
5′ GATCCCGTTGTTAGGCA 3′
and
17-mer reverse primer
(SEQ ID NO: 17)
5′ TCGATGACCACACCATG 3′,
and
a labeled 14-mer probe
(SEQ ID NO: 18)
5′ 6-FAM-GTGTGTGCACAGTC-MGB-NFQ 3′;
and
(f)
a 21-mer oligonucleotide forward primer
(SEQ ID NO: 19)
5′ ATTCTCTGCTATTTGGTGACG 3′
and
17-mer reverse primer
(SEQ ID NO: 20)
5′ ACCTTCAGCAACAAGGC 3′,
and
a labeled 26-mer probe
(SEQ ID NO: 21)
5′ LC670-ATGCCCATAGCCAAAGAGAGTCCACG-BBQ 3′.

14. A method for detecting at least one or more intestinal parasites selected from Dipylidium caninum, Toxocara canis, Ancylostoma caninum, Cystoisospora canis, Trichuris vulpis and Giardia duodenalis from a sample taken from a canine subject and treating the canine subject, the method comprising:
(a) providing a sample comprising biological material taken from the subject or nucleic acids extracted from the biological material taken from the subject, wherein the sample includes at least 0.1 femtograms of nucleic acids;
(b) providing a composition suitable for detecting the one or more intestinal parasites selected from Dipylidium caninum, Toxocara canis, Ancylostoma caninum, Cystoisospora canis, Trichuris vulpis and Giardia duodenalis, the composition comprising one or more of an oligonucleotide primer pair comprising forward and reverse primers directed to repetitive elements of Dipylidium caninum, Toxocara canis, Ancylostoma caninum, Cystoisospora canis, Trichuris vulpis or Giardia duodenalis;
(c) contacting the sample with the composition of step (b) under conditions suitable for specific hybridization and extension of hybridized nucleic acids by polymerase chain reaction (PCR) to produce one or more PCR extension products, each PCR extension product associated with Dipylidium canimum, Toxocara canis, Ancylostoma caninum, Cystoisospora canis, Trichuris vulpis or Giardia duodenalis;
(d) detecting for the one or more PCR extension products as an indication of the presence or absence of one or more intestinal parasites selected from Dipylidium caninum, Toxocara canis, Ancylostoma caninum, Cystoisospora canis, Trichuris vulpis and Giardia duodenalis; and
(e) determining the presence of Dipylidium caninum in the sample if a PCR extension product associated with Dipylidium caninum is detected; the presence of Toxocara canis in the sample if a PCR extension product associated with Toxocara canis is detected; the presence of Ancylostoma caninum in the sample if a PCR extension product associated with Ancylostoma caninum is detected; the presence of Cystoisospora canis in the sample if a PCR extension product associated with Cystoisospora canis is detected; the presence of Trichuris vulpis in the sample if a PCR extension product associated with Trichuris vulpis is detected; and/or the presence of Giardia duodenalis in the sample if a PCR extension product associated with Giardia duodenalis is detected, and
(f) if the presence of one or more Dipylidium caninum, Toxocara canis, Ancylostoma caninum, Cystoisospora canis, Trichuris vulpis and/or Giardia duodenalis is detected, administering to the canine subject a treatment effective at reducing or eliminating parasite infection.

15. A kit for detecting one or more intestinal parasites selected from Dipylidium caninum, Toxocara canis, Ancylostoma canimum, Cystoisospora canis, Trichuris vulpis and Giardia duodenalis from a sample taken from a canine subject, the kit comprising:
(a) one or more oligonucleotide primer pairs comprising of forward and reverse primers directed to repetitive elements of Dipylidium caninum, Toxocara canis, Ancylostoma canimum, Cystoisospora canis, Trichuris vulpis or Giardia duodenalis;
(b) detection probes for detecting PCR extension products associated with the one or more intestinal parasites selected from Dipylidium canimum, Toxocara canis, Ancylostoma canimum, Cystoisospora canis, Trichuris vulpis and Giardia duodenalis; and
(c) optional buffers and a set of instructions.

16. The kit according to claim 14, wherein parts (i) and (ii) are part of an oligonucleotide primer/probe set, the oligonucleotide primer/probe set selected from the group consisting of:
(a)
a 17-mer oligonucleotide forward primer
(SEQ ID NO: 1)
5′ GGCACCTGTCTGTCAGG 3′
and
19-mer reverse primer
(SEQ ID NO: 2)
5′ TCTAAGCGTCTGCAATTCG 3′,
a labeled 27-mer donor probe
(SEQ ID NO: 3)
5′ ACGTTTAATGTTTGCAGAATCGTGACT-FL 3′
and
a labeled 24-mer acceptor probe
(SEQ ID NO: 4)
5′ LC640-ACCTAGCTTCAGCGATGGATCGGT-PH 3′;
(b)
a 17-mer oligonucleotide forward primer
(SEQ ID NO: 5)
5′ TTCCGAACGGCGGATCA 5′
and
16-mer reverse primer
(SEQ ID NO: 6)
5′ CTCAGACAGGCGTAGC 3′,
a labeled 30-mer donor probe
(SEQ ID NO: 7)
5′ TGATGTGAATTGCAGACACACTGAACTTGA-FL 3′
and
a labeled 26-mer acceptor probe
(SEQ ID NO: 8)
5′ LC610-TACTTTGAACGCACATTGCAGCGTCG-PH 3′;
(c)
a 21-mer oligonucleotide forward primer
(SEQ ID NO: 9)
5′ TCTTGCATTTGTGGTTGCCTA 3′
and
22-mer reverse primer
(SEQ ID NO: 10)
5′ GCATCACTCTGAAATACACAAC 3′,
labeled 16-mer donor probe
(SEQ ID NO: 11)
5′ GTTGAGGCCCCCACGA-FL 3′
and
labeled 24-mer acceptor probe
(SEQ ID NO: 12)
5′ LC670-CCAGTATGTTGTTGGCTGGTCTTT-PH 3′;
(d)
a 26-mer oligonucleotide forward primer
(SEQ ID NO: 13)
5′ CTTTTAGAAGATGATTACCTAAGGCT 3′
and
21-mer reverse primer
(SEQ ID NO: 14)
5′ ACGTTCTACTATGAACCAACG 3′,
and
a 20-mer probe
(SEQ ID NO: 15)
5′ LC640-AGTGCGCCGACGCCTGTTAG-BBQ 3′;
(e)
a 17-mer oligonucleotide forward primer
(SEQ ID NO: 16)
5′ GATCCCGTTGTTAGGCA 3′
and
17-mer reverse primer
(SEQ ID NO: 17)
5′ TCGATGACCACACCATG 3′,
and
a labeled 14-mer probe
(SEQ ID NO: 18)
5′ 6-FAM-GTGTGTGCACAGTC-MGB-NFQ 3′;
and
(f)
a 21-mer oligonucleotide forward primer
(SEQ ID NO: 19)
5′ ATTCTCTGCTATTTGGTGACG 3′
and
17-mer reverse primer
(SEQ ID NO: 20)
5′ ACCTTCAGCAACAAGGC 3′,
and
a labeled 26-mer probe
(SEQ ID NO: 21)
5′ LC670-ATGCCCATAGCCAAAGAGAGTCCACG-BBQ 3′.

17. A oligonucleotide primer/probe set selected from the group consisting of:
(a)
a 17-mer oligonucleotide forward primer
(SEQ ID NO: 1)
5′ GGCACCTGTCTGTCAGG 3′
and
19-mer reverse primer
(SEQ ID NO: 2)
5′ TCTAAGCGTCTGCAATTCG 3′,
a labeled 27-mer donor probe
(SEQ ID NO: 3)
5′ ACGTTTAATGTTTGCAGAATCGTGACT-FL 3′
and
a labeled 24-mer acceptor probe
(SEQ ID NO: 4)
5′ LC640-ACCTAGCTTCAGCGATGGATCGGT-PH 3′;
(b)
a 17-mer oligonucleotide forward primer
(SEQ ID NO: 5)
5′ TTCCGAACGGCGGATCA 5′
and
16-mer reverse primer
(SEQ ID NO: 6)
5′ CTCAGACAGGCGTAGC 3′,
a labeled 30-mer donor probe
(SEQ ID NO: 7)
5′ TGATGTGAATTGCAGACACACTGAACTTGA-FL 3′
and
a labeled 26-mer acceptor probe
(SEQ ID NO: 8)
5′ LC610-TACTTTGAACGCACATTGCAGCGTCG-PH 3′;
(c)
a 21-mer oligonucleotide forward primer
(SEQ ID NO: 9)
5′ TCTTGCATTTGTGGTTGCCTA 3′
and
22-mer reverse primer
(SEQ ID NO: 10)
5′ GCATCACTCTGAAATACACAAC 3′,
labeled 16-mer donor probe
(SEQ ID NO: 11)
5′ GTTGAGGCCCCCACGA-FL 3′
and
labeled 24-mer acceptor probe
(SEQ ID NO: 12)
5′ LC670-CCAGTATGTTGTTGGCTGGTCTTT-PH 3′;
(d)
a 26-mer oligonucleotide forward primer
(SEQ ID NO: 13)
5′ CTTTTAGAAGATGATTACCTAAGGCT 3′
and
21-mer reverse primer
(SEQ ID NO: 14)
5′ ACGTTCTACTATGAACCAACG 3′,
and
a 20-mer probe
(SEQ ID NO: 15)
5′ LC640-AGTGCGCCGACGCCTGTTAG-BBQ 3′;
(e)
a 17-mer oligonucleotide forward primer
(SEQ ID NO: 16)
5′ GATCCCGTTGTTAGGCA 3′
and
17-mer reverse primer
(SEQ ID NO: 17)
5′ TCGATGACCACACCATG 3′,
and
a labeled 14-mer probe
(SEQ ID NO: 18)
5′ 6-FAM-GTGTGTGCACAGTC-MGB-NFQ 3′;
and
(f)
a 21-mer oligonucleotide forward primer
(SEQ ID NO: 19)
5′ ATTCTCTGCTATTTGGTGACG 3′
and
17-mer reverse primer
(SEQ ID NO: 20)
5′ ACCTTCAGCAACAAGGC 3′,
and
a labeled 26-mer probe
(SEQ ID NO: 21)
5′ LC670-ATGCCCATAGCCAAAGAGAGTCCACG-BBQ 3′.


SEQUENCE LISTING (21 sequences)

SEQ ID NO: 1 - DNA, 17 bp, synthetic construct
  source 1..17: mol_type = other DNA
ggcacctgtctgtcagg

SEQ ID NO: 2 - DNA, 27 bp, synthetic construct
  source 1..27: mol_type = other DNA
acgtttaatgtttgcagaatcgtgact

SEQ ID NO: 3 - DNA, 27 bp, synthetic construct
  source 1..27: mol_type = other DNA
acgtttaatgtttgcagaatcgtgact

SEQ ID NO: 4 - DNA, 24 bp, synthetic construct
  source 1..24: mol_type = other DNA
acctagcttcagcgatggatcggt

SEQ ID NO: 5 - DNA, 17 bp, synthetic construct
  source 1..17: mol_type = other DNA
ttccgaacggcggatca

SEQ ID NO: 6 - DNA, 16 bp, synthetic construct
  source 1..16: mol_type = other DNA
ctcagacaggcgtagc

SEQ ID NO: 7 - DNA, 30 bp, synthetic construct
  source 1..30: mol_type = other DNA
tgatgtgaattgcagacacactgaacttga

SEQ ID NO: 8 - DNA, 26 bp, synthetic construct
  source 1..26: mol_type = other DNA
tactttgaacgcacattgcagcgtcg

SEQ ID NO: 9 - DNA, 21 bp, synthetic construct
  source 1..21: mol_type = other DNA
tcttgcatttgtggttgccta

SEQ ID NO: 10 - DNA, 22 bp, synthetic construct
  source 1..22: mol_type = other DNA
gcatcactctgaaatacacaac

SEQ ID NO: 11 - DNA, 16 bp, synthetic construct
  source 1..16: mol_type = other DNA
gttgaggcccccacga

SEQ ID NO: 12 - DNA, 24 bp, synthetic construct
  source 1..24: mol_type = other DNA
ccagtatgttgttggctggtcttt

SEQ ID NO: 13 - DNA, 26 bp, synthetic construct
  source 1..26: mol_type = other DNA
cttttagaagatgattacctaaggct

SEQ ID NO: 14 - DNA, 21 bp, synthetic construct
  source 1..21: mol_type = other DNA
acgttctactatgaaccaacg

SEQ ID NO: 15 - DNA, 20 bp, synthetic construct
  source 1..20: mol_type = other DNA
agtgcgccgacgcctgttag

SEQ ID NO: 16 - DNA, 17 bp, synthetic construct
  source 1..17: mol_type = other DNA
gatcccgttgttaggca

SEQ ID NO: 17 - DNA, 17 bp, synthetic construct
  source 1..17: mol_type = other DNA
tcgatgaccacaccatg

SEQ ID NO: 18 - DNA, 14 bp, synthetic construct
  source 1..14: mol_type = other DNA
gtgtgtgcacagtc

SEQ ID NO: 19 - DNA, 21 bp, synthetic construct
  source 1..21: mol_type = other DNA
attctctgctatttggtgacg

SEQ ID NO: 20 - DNA, 17 bp, synthetic construct
  source 1..17: mol_type = other DNA
accttcagcaacaaggc

SEQ ID NO: 21 - DNA, 26 bp, synthetic construct
  source 1..26: mol_type = other DNA
atgcccatagccaaagagagtccacg